SEQUENCE LISTING (4 sequences)

SEQ ID NO: 1 - DNA, 11405 bp, Artificial sequence
  vector
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagttagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactagctcagatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagactcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctctatttttacaacaattaccaacaacaacaaacaacaaacaacattacaattacatttacaattaccatggggcgcgcccaatcgatgatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacantttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtnaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtatttggatcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 2 - DNA, 3367 bp, Artificial sequence
  vector
ggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagccccaaacacacgctcggacgcatattacacatgttcatacacttaatactcgctgttttgaattgatgttttaggaatatatatgtagagagagcttccttgagtccattcacaggtcgtgatatgattcaattagcttccgactcattcatccaaataccgagtcgccaaaattcaaactagactcgttaaatgaagaatgatgcggtagacaaattggatcattgattctctttgattggactgaagggagctccctctctcttttgtattccaattttcttgattaatctttcctgcacaaaaacatgcttgatccactaagtgacatatatgctgccttcgtatatatagttctggtaaaattaacattttgggtttatctttatttaaggcatcgccatggggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 3 - DNA, 18691 bp, Artificial sequence
  vector
ggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgaggctagcatggatctcgggccccaaataatgattttattttgactgatagtgacctgttcgttgcaacaaattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtagtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctcctcgaggaattcggtaccccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatacggccggtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatcgcgtggatccggcttactaaaagccagataacagtatgcgtatttgcgcgctgatttttgcggtataagaatatatactgatatgtcgggcccataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaaattgggttcgaaatcgataagcttggatcctctagagagctgcagctggatggcaaataatgattttattttgactgatagtgacctgttcgttgcaacaaattgataagcaatgctttcttataatgccaactttgtacaagaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgactaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcactacccatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttctgttcttgatgcagatgattttcaggactatgacactagcgtatatgaataggtagatgtttttattttgtcacacaaaaaagaggctcgcacctctttttcttatttctttttatgatttaatacggcattgaggacaatagcgagtaggctggatacgacgattccgtttgagaagaacatttggaaggctgtcggtcgactaagttggcagcatcacccgaagaacatttggaaggctgtcggtcgactacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttggggcccgagatccatgctagctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatctgc

SEQ ID NO: 4 - DNA, 12096 bp, Artificial sequence
  vector
aattcagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctccaccgcggtgggcggccgctctagaactagttaattaaggaattattaccctttgatcctgaatagagcactgccatctgaaacctgttggcctgctttaactttgaggtcctgtatgcttccagaggatggtgctttcacaacgtgctccatcttcattgcctctaggactaatataggttgaccttgatctactttggcttcgttttcgacaaggaccttgactaccaaaccagccatgggggccacaatggttcctggagggtgtgatgatgtttcggagctggttggatctgcggaacccagtggaccctggaacaaaacttcgaggccaccggatgcataatcaggaacgtcataagggtaagaacctgtgcggccgccgctagctcgaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtttgatagcttggcgcgcctcgagggggggcccggtacccggggatcctctagagtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcacgccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcaaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

(19) United States
(12) Patent Application Publication
(10) Pub. No.: US 2016/0208282 A1
(43) Pub. Date: Jul. 21, 2016

(54) SYSTEMS FOR CLONING PLANTS THROUGH ASEXUAL MEANS

(71) Applicant: CENTRO DE INVESTIGACION Y ESTUDIOS AVANZADOS DEL INSTITUTO POLITECNICO NACIONAL (CINVESTAV), Mexico D.F. (MX)

(72) Inventors: Jean-Philippe Vielle Calzada, Irapuato Guanajuato (MX); Carlos Barragan Rosillo, Irapuato Guanajuato (MX); Edgar Demesa Arevalo, Irapuato Guanajuato (MX); Elvira Hernandez Lagana, Irapuato Guanajuato (MX); Gloria Leon Martinez, Irapuato Guanajuato (MX); Nidia Sanchez Leon, Irapuato Guanajuato (MX); Daniel Rodriguez Leal, Irapuato Guanajuato (MX); Isaac Rodriguez Arevalo, Irapuato Guanajuato (MX); Jaime Padilla Calzada, Irapuato Guanajuato (MX)

(21) Appl. No.: 15/023,950

(22) PCT Filed: Sep. 22, 2014
(86) PCT No.: PCT/IB2014/002702

§ 371 Date: Mar. 22, 2016

Related U.S. Application Data
(60) Provisional application No. 61/881,345, filed on Sep. 23, 2013.

(51) Int. Cl. C12N 15/82; A01H 4/00; A01H 1/02

(52) U.S. Cl. CPC C12N 15/8287; C12N 15/8261; A01H 1/02; C12N 15/8218; A01H 4/006

(57) ABSTRACT

Disclosed are methods of obtaining clonal seeds, methods of plant cloning, methods of screening for maternal plants that produce clonal seeds asexually and methods of increasing yield of clonal seeds. Also disclosed are constructs comprising a nucleic acid that can silence the activity of a RNA-dependent DNA methylation pathway gene. Further disclosed are maternal plants comprising a construct wherein the construct comprises an exogenous nucleic acid sequence, wherein the construct renders the maternal plant defective for RNA-dependent DNA methylation.

### CROSS-REFERENCE TO RELATED APPLICATIONS

This application is a National Phase Under 35 U.S.C. §371 of PCT/IB2014/002702 filed in the Patent Cooperation Treaty U.S. Receiving Office on Sep. 22, 2014, which claims the benefit of U.S. Provisional Application No. 61/881,345, filed Sep. 23, 2013, the entire contents of which are herein incorporated by reference.

### REFERENCE TO SEQUENCE LISTING

The sequence listing submitted Mar. 22, 2016 as a text file named “37487_0001U2_Sequence_Listing.txt,” created on Mar. 22, 2016, and having a size of 60,374 bytes is hereby incorporated by reference pursuant to 37 C.F.R. §1.52(e)(5).

### BACKGROUND

The majority of cultivated plants reproduce in a sexual manner. In sexual reproduction, the fusion of male and female gametes leads to the formation of seeds that combine maternal and paternal traits.

Although sexual reproduction predominates, plant species exist that reproduce themselves asexually through seeds. This asexual reproduction, termed apomixis, is a natural cloning process by which the female reproductive organ of a plant, the ovule, is able to form the embryonic portion of seeds, without the need for a genetic contribution from male gametes. In particular, an ovule of an apomictic plant produces one or more unreduced female gametes that form without undergoing meiosis. Accordingly, each unreduced female gamete maintains the somatic genotype of the parent plant when the gamete is incorporated into a seed and ultimately develops to form a child plant that is a clone of the parent.

The induction of apomixes in cultivated plants, such as in edible cereals, constitutes one of the most attractive challenges of agricultural biotechnology. Currently, the majority of improved, commercial seeds are the result of a long hybridization process in which certain plants that present desirable traits are selected and crossed to obtain seeds for an improved hybrid. However, the agronomic value of the improved hybrid is maintained only during one cultivation cycle. The natural sexuality of the hybrid causes the next generation to lose many of the desirable characteristics of the hybrid through separation of genetic traits. As a consequence, competitive producers find themselves obliged to buy seed year after year if they want to maintain high performance.

The ability to generate apomictic plant varieties would have tremendous commercial benefits. For example, creation of improved hybrids that exhibit a high rate of apomixis may, in some cases, make it possible for farmers to recurrently sow the seed produced by the improved hybrid, thereby maintaining the agronomic value of the seed for multiple generations (and potentially indefinitely). Also, by genetically fixing the agronomic value of any sexual cultivation, the ability to induce apomixis may encourage plant breeders to develop customized plant varieties adapted to specific environmental conditions. Additionally, the induction of apomixis offers the possibility of eliminating the use of costly cultivation techniques associated with vegetative reproduction of crop plants (e.g., potato, agave, and strawberry, among others). An ability to induce apomixis also may permit the preservation of individual plants with high rates of heterozygosis, such as vegetable species that are in danger of extinction.

Thus, there is a need for a maternal plant that produces clonal seeds asexually, with each clonal seed containing an embryo that is a clone of the maternal plant, and with germination of each clonal seed forming a progeny plant that is a clone of the maternal plant.

### BRIEF SUMMARY

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant.

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant, wherein the maternal plant is defective in at least one RNA dependent DNA methylation pathway gene. For example, the RNA dependent DNA methylation pathway gene can be AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NRPD1b (NUCLEAR POLYMERASE D 1b), NRPD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9). In some instances, the AGO4 allele can be ago4-6 or ago4-1. In some instances, the AGO6 allele can be ago6-2. In some instances, the AGO9 allele can be 9-2, 9-3 or 9-4. In some instances, the AGO8 allele can be ago 8-1. In some instances, the RDR2 allele can be rdr2-1. In some instances, the RDR6 allele can be rdr6-15 or rdr6-11. In some instances, the SGS3 allele can be sgs3-11. In some instances, the DRM2 allele can be drm2-2. In some instances, the MET1 allele can be met1-7.

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant further comprising pollinating the maternal plant prior to collecting the seeds and sorting the seeds to separate clonal seeds from non-clonal seeds.

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant further comprising pollinating the maternal plant prior to collecting the seeds and sorting the seeds to separate clonal seeds from non-clonal seeds, wherein sorting the seeds is based on distinguishing the size, shape, size and shape, or genetics of the embryos. The sorting can be performed manually or automatically. In some instances, automatic sorting comprises a machine comprising an optical detector. In some instances, the sorting can be done visually.

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant, wherein the maternal plant is exposed to a gametocide that abolishes pollen formation. Gametocides can include at least one of maleic hydrazide (1,2-dihydropyridazine, 3-6-dione) (MH), 2,4-dichlorophenoxyacetic acid (2,4-D), a-naphthalene acetic acid (NAA), and tri-iodobenzoic acid (TIBA).

Disclosed are methods of obtaining clonal seeds comprising a) obtaining a maternal plant, wherein the maternal plant is unable to be pollinated; and b) collecting one or more seeds produced by the maternal plant, wherein the one or more seeds comprise an embryo that is a clone of the maternal plant further comprising emasculating the maternal plant before collecting the seeds.

Also disclosed are methods of screening for maternal plants that produce clonal seeds asexually comprising a) obtaining a maternal plant; b) silencing the activity of a gene of interest producing a transformed maternal plant; crossing the transformed maternal plant with a sterile male plant; and d) harvesting the seeds; wherein the presence of clonal seeds indicates the maternal plant can produce clonal seeds asexually.

Disclosed are methods of screening for maternal plants that produce clonal seeds asexually comprising a) obtaining a maternal plant; b) silencing the activity of a gene of interest producing a transformed maternal plant; crossing the transformed maternal plant with a sterile male plant; and d) harvesting the seeds; wherein the presence of clonal seeds indicates the maternal plant can produce clonal seeds asexually, wherein silencing the activity of a gene of interest comprises RNA interference.

Disclosed are methods of screening for maternal plants that produce clonal seeds asexually comprising a) obtaining a maternal plant; b) silencing the activity of a gene of interest producing a transformed maternal plant; crossing the transformed maternal plant with a sterile male plant; and d) harvesting the seeds; wherein the presence of clonal seeds indicates the maternal plant can produce clonal seeds asexually, wherein the gene of interest is a RNA dependent DNA methylation pathway gene. The gene of interest can be AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

Disclosed are methods of increasing the yield of clonal seeds comprising obtaining a maternal plant; pollinating the maternal plant; collecting a mixture of seeds produced by the maternal plant; and sorting the mixture to separate clonal seeds from non-clonal seeds.

Disclosed are methods of increasing the yield of clonal seeds comprising obtaining a maternal plant; pollinating the maternal plant; collecting a mixture of seeds produced by the maternal plant; and sorting the mixture to separate clonal seeds from non-clonal seeds, wherein sorting the mixture to separate clonal seeds from non-clonal seeds comprises distinguishing clonal embryos from non-clonal embryos. Distinguishing clonal embryos from non-clonal embryos can comprise determining the size, shape, size and shape, or genetics of the embryos. In some instances, the sorting can be performed manually or automatically. Automatic sorting can comprise a machine comprising an optical detector. In some instances, sorting can be done visually.

Disclosed are methods of increasing the yield of clonal seeds comprising obtaining a maternal plant; pollinating the maternal plant; collecting a mixture of seeds produced by the maternal plant; and sorting the mixture to separate clonal seeds from non-clonal seeds, wherein the maternal plant is defective in at least one RNA dependent DNA methylation pathway gene. RNA dependent DNA methylation pathway genes can be AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

Disclosed are maternal plants comprising a construct, wherein the construct comprises an exogenous nucleic acid sequence, wherein the construct renders the maternal plant defective for RNA-dependent DNA methylation.

Disclosed are maternal plants comprising a construct, wherein the construct comprises an exogenous nucleic acid sequence, wherein the construct renders the maternal plant defective for RNA-dependent DNA methylation, wherein the exogenous nucleic acid sequence silences activity of a RNA-dependent DNA methylation pathway gene.

Disclosed are maternal plants comprising a construct, wherein the construct comprises an exogenous nucleic acid sequence, wherein the construct renders the maternal plant defective for RNA-dependent DNA methylation, wherein the exogenous nucleic acid sequence silences activity of a RNA-dependent DNA methylation pathway gene further comprising a clonal seed.

Also disclosed are maternal plants comprising a defective RNA-dependent DNA methylation pathway gene.

Disclosed are maternal plants comprising a defective RNA-dependent DNA methylation pathway gene, wherein the RNA dependent DNA methylation pathway gene is AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows that homozygous mutant individuals of rdr6-15, ago4-1, and agog-3 form viable 2n gametes. Flow cytometry was used to estimate the number of triploid progeny that was recovered; triploid seeds are usually larger than diploid seeds. In all three cases, triploid progeny was recovered.

FIG. 2 shows seed formation in unpollinated siliques of heterozygous rdr6-15 plants. In contrast to unpollinated pistillata gynoecia (A), unpollinated siliques of rdr16-15/+ individuals show a large frequency of growing seeds (B and C). (D) Frequency of turgid developing seed-like organs in unpollinated siliques of pi, F2 pi rdr6-15/+ (Group A), and F3 pi rdr6-15/+ (Group B) individuals. (E-G) Whole-mount cleared seeds contain free nuclear endosperm and normally organized embryos.

FIG. 3 shows autonomous seed formation in emasculated plants of ago4-5, agog-2 and rdr6-15 plants. (A) embryo from a rdr6-15 autonomous developing seed. (B and C) embryos from ago4-6 autonomous seeds; uncellularized endosperm nuclei are marked by an arrow. (D to E) Autonomous seeds of ago9-2 showing an early globular embryo (dashed) with embryo-proper (Emb) and suspensor (S). (G to H) show vanillin stain in the micropylar region of a young autonomous developing seed. A and F, scale bar=50 μm; B, E, G and H, scale bar=12.5 μm; C and D, scale bar=25 μm.

FIG. 4 shows that plants originating from mature seeds produced in the absence of pollination are genetically equivalent to their mother. Maternal individuals and their progeny were genotyped for polymorphic loci; each row represents a plant and each column is a locus.

FIG. 5 shows the quantification of ploidy levels and seed recovery in nonpollinated pi individuals carrying mutations in rdr6 or ago4.

FIG. 6 shows ectopic gametic precursors and gametophytes in homozygous ago4 individuals. Several ectopic pre-meiotic cells differentiate in the young ovule primordial (A), resulting in several developing female gametophytes ectopically ocated at the micropylar and chalazal regions of the ovule (B).

FIG. 7 shows emasculated inflorescences and siliques bearing developing seeds. A-C scale bar=1 mm; D-F scale bar=0.5 mm.

FIG. 8 shows the frequency of turgid and aborted seeds and ovules in pi and pi rdr6-15/+F2 and F3 individuals. S4 refers to the 4th siliques top to bottom (S1 being the first gynoecia within the inflorescence that has completely lost its floral organs following floral senescence); S20 is the oldest unpollinated silique.

FIG. 9 provides general data of emasculated rdr6-15, ago4-6, and ago9-2 mutants.

FIG. 10 shows the individual analysis of emasculated ago9-2 plants.

FIG. 11 shows the individual analysis of emasculated rdr6-15 plants.

FIG. 12 shows the individual analysis of emasculated ago4-6 plants.

FIG. 13 shows the quantification of ploidy levels and seed recovery in nonpollinated pi individuals carrying the mutations pistillata and rdr6-15 or ago4-1. Screening using a male sterile background showed the potential of small RNA mutants for autonomous seed formation.

FIG. 14 shows that genotyping using 89 SNPs markers proved that part of the recovered progeny was clonal. The parental lines and progeny were confirmed to be 2n by flow cytometry.

FIG. 15 shows that individuals showed variation in one or two out of the 89 SNPs with respect to its parental line. The parental lines and progeny were confirmed to be 2n by flow cytometry.

FIG. 16 shows that ovules in double mutants (pi rdr-15) were long-lived compared to pistillata controls and exhibited seed-like features.

FIG. 17 shows that ago9-2, ago9-3, ago4-6, and rdr6-15/+ exhibited a higher frequency of long-lived ovules after 7, 10 and 14 Days After Emasculation (DAE) compared to wild-type plants. The delay of ovule degeneration in the mutant backgrounds could be indicative of the activation of an autonomous seed formation program.

FIG. 18 shows the percentage of seeds recovered from ago4-6, ago9-2, rdr6-15/+, and wild type plants at 7DAE.

FIG. 19 shows the seeds recovered from fully-dried emasculated carpels of more than 30 DAE.

FIG. 20 shows the seed coat formation in non-fertilized ovules. Detailed cytological analysis of non-pollinated wild type and mutant ovules after 7, 10 and 14 DAE confirmed that ago9, ago4, and rdr6 single mutants initiate autonomous seed formation.

FIG. 21 shows proanthocyanidin accumulation in the endothelium in Col-0, rdr6-15/+, ago9-2, ago9-3, and ago4-6 mutants. Vanilin red staining, as indicated by the white dashed circles, is positive for the presence of proanthocyanidins.

FIG. 22 shows that all three mutants, rdr6-15, ago9-3 and ago4-6, are able to form autonomous endosperms in the absence of pollination.

FIG. 23 shows that ago9 ovules show premature and higher frequency of autonomous endosperm proliferation than ago4 or rdr6.

FIG. 24 shows that autonomous embryo development was also detected at low frequencies.

FIG. 25 provides a complete list of mutants and alleles that show ectopic gametic precursor cells reminiscent of aposporous initials (apomixis).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a maternal plant is disclosed and discussed and a number of modifications that can be made are discussed, each and every combination and permutation of the maternal plant and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. DEFINITIONS

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the polypeptide” is a reference to one or more polypeptide and equivalents thereof known to those skilled in the art, and so forth. Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The term “exogenous” means, introduced from or produced outside the organism or system, for example, a promoter sequence that is sourced from an organism that is of different genetic origin than the organism it is introduced into is an exogenous sequence.

“Parthenogenically-derived embryo” refers to an embryo that develops autonomously, i.e. without the need of fusion of a female and male gamete, or their corresponding DNA.

“Sexually-generated embryos” refer to embryos that contain both male and female DNA. Sexually-generated embryos are also referred to as non-clonal embryos.

The phrase “unreduced gamete” refers to a reproductive cell formed by a plant, having the same (“unreduced”) ploidy and/or genotype as somatic (sporophyte) cells of the plant, and capable of contributing genetic material for embryo formation. The gamete can be formed by and/or present in an ovule of the plant and can be described as a female gamete, whether or not the gamete is capable of uniting with a male gamete. A diploid plant produces unreduced gametes that are diploid, a triploid plant produces unreduced gametes that are triploid, as so on. An unreduced female gamete can unite with a male gamete to form a zygote that develops into an embryo, or, in some cases, can develop into an embryo without uniting with a male gamete. An unreduced female gamete can be described as having the same genotype as somatic cells of the plant, which means that at least substantially every allele of a somatic cell is also present in the gamete. In some examples, the chromosomal constitution of the gamete (or of a progeny plant or next generation) can be described as a somatic chromosomal constitution, which means that a copy of each and every somatic chromosome of the parent plant is present in the gamete (or child plant or next generation), with the linkage of alleles on each individual chromosome preserved when comparing somatic cells of the parent plant to the gamete (or child plant or next generation). A somatic chromosomal constitution can be generated in a gamete when no segregation or recombination occurs between homologous chromosomes during gamete formation.

Unreduced female gametes can be formed by diplospory or apospory, among others. The process of diplospory generates an unreduced gamete from a typical gamete precursor, a megaspore mother cell (MMC), which fails to undergo meiosis. The process of apospory generates an unreduced gamete by direct differentiation of a somatic cell into a gamete precursor, an MMC-like cell. The MMC-like cell generally is formed in a distinct site from the MMC (if present). Apospory can occur via a supernumerary gamete precursor while the usual gamete precursor undergoes meiosis (or apomeiosis).

Unreduced female gametes can be generated at any suitable frequency relative to total female gametes (unreduced and meiotically reduced). For example, the frequency of unreduced female gametes generated by an individual plant can be at least about 1%, 5%, 10%, or 25%, among others.

The term “apomixis” (plural apomixes) refers to clonal reproduction through seeds. Apomixis is the process by which a maternal plant produces one or more clonal seeds each containing an embryo that is a clone of the maternal plant. In other words, the embryo is genetically identical to the maternal plant and contains no genetic material from a paternal plant (e.g., a diploid maternal plant produces a diploid embryo clone). The embryo clone contains the unreduced genome (the somatic genome) of the maternal plant. Each of the clonal seeds interchangeably can be termed an asexual seed or an apomictic seed. The clonal seed can contain endosperm that is produced without plant pollination (i.e., the endosperm contains only the maternal genome and no paternal genes/genome). Alternatively, if the maternal plant has been pollinated, the endosperm of the clonal seed can result from fertilization and can have a maternal contribution and a paternal contribution (from pollen). Each clonal seed, if viable, can germinate to produce a progeny plant that is a clone of the maternal plant. A maternal plant that reproduces by apomixis forms viable clonal seeds at a detectable frequency, with any suitable percentage of its seeds being clonal, such as at least about 0.1%, 0.2%, 0.3%, 0.4%, 0.5%, 0.6%, 0.7%, 0.8%, 0.9%, 1%, 5%, 10%, 20%, 50%, or 100%, among others. The number and frequency of clonal seeds and non-clonal seeds can be influenced by the genotype of the maternal plant and its environment (i.e., whether or not the maternal plant is pollinated).

Apomixis can occur in a maternal plant when an RNA-dependent DNA methylation (RdDM) pathway is defective. Accordingly, apomixis can be induced by rendering the pathway defective. The pathway may be involved in silencing repetitive sequences by methylation of the sequences. RdDM can be required to prevent apomixis. The structure, expression, and/or activity of at least one member of the RdDM pathway can be altered to render RdDM defective and promote apomixis. The member(s) can be one or more of AGO4, AGO6, AGO9, CMT3, DCL3, DRM2, IDS2, MET1, NPRD1a, NPRD1b, NRPE1, NRPE2, RDR2, RDR6, SGS3, SUVH2, and SUVH9, among others, or a functional/structural homolog of any of the members from a different species. The homolog can exhibit homology through identity or similarity at the gene, RNA, and/or polypeptide level.

An amount of identity or similarity between two polypeptides may be determined by the blastp algorithm (e.g., program BLASTP 2.2.18+), as described in the following two references, which are incorporated herein by reference: Stephen F. Altschul, et al. (1997), “Gapped BLAST and PSI-BLAST: a new generation of protein database search programs,” Constructs Res. 25:3389-3402; and Stephen F. Altschul et al. (2005) “Protein database searches using compositionally adjusted substitution matrices,” FEBS J. 272:5101-5109. Examples of substantial similarity or identity include at least about 40%, 50%, 60%, 70%, or 80% sequence similarity or identity, a similarity score of at least about 200 or 250, and/or an E-Value of less than about 1e-40, 1e-60, or 1e-80, among others, using the blastp algorithm, with optimal alignment and, if needed, introduction of gaps.

A DNA sequence is “operatively linked” to an expression control sequence when the expression control sequence controls and regulates the transcription and translation of that DNA sequence. For example, the DNA sequence can be a targeting region and the expression control sequence can be a promoter. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence.

The phrase “RNA interference” refers to a process of inhibiting gene expression in a targeted fashion using RNA mediators, which can be termed interfering RNAs. Interfering RNAs may include double-stranded RNAs, short interfering RNAs, micro RNAs, and/or the like. In some embodiments, the interfering RNA, as expressed or introduced, can be a double-stranded RNA, such as an RNA with a hairpin structure, which can be processed in the cell to form a small RNA (e.g., a short interfering RNA or a micro RNA). Small RNAs generally include RNAs of less than about 30 nucleotides, such as RNAs of 20, 21, 22, 23, 24, or 25 nucleotides, among others. RNA interference can inhibit gene expression before, during, and/or after transcription of a gene (i.e., by a transcriptional and/or a post-transcriptional mechanism), such as by gene modification (e.g., DNA/histone methylation), mRNA degradation, and/or inhibition of mRNA translation, among others.

The phrase “optionally being defective for RNA-dependent DNA methylation” means that a plant can be defective for RNA-dependent DNA methylation. Being defective for RNA-dependent DNA methylation means that at least one gene in the RNA-dependent DNA methylation pathway has been altered to the extent that the altered gene results in defective RNA-dependent DNA methylation. In some instances, more than one gene in the RNA-dependent DNA methylation pathway can be altered, wherein the combination of the more than one altered genes results in defective RNA-dependent DNA methylation. An altered gene that results in defective RNA-dependent DNA methylation can include an alteration (substitution or deletion) or modification to any gene that encodes a product involved in and/or required for RNA-dependent DNA methylation, wherein the alteration or modification result in defective RNA-dependent DNA methylation.

The term “plant” refers to a member of the Plantae kingdom of eukaryotic organisms, which can be described as a tree, bush, grass, shrub, herb, vine, moss, fern, algae, or a combination thereof, among others. A plant may (or may not) lack the capability for locomotive movement and generally possesses cell walls formed of cellulose. A plant may be capable of carrying out photosynthesis and may (or may not) be a vascular plant. In some embodiments, the plant can be an annual or a perennial. The plant can be a flowering plant (an angiosperm), such as a monocotyledon or a dicotyledon. In some embodiments, the plant can produce a grain, tuber, fruit, vegetable, nut, seed, fiber, oil, or a combination thereof, among others. Furthermore, the plant can be a crop plant. Exemplary crop plants that can be suitable for generation of transgenic plants according to the present disclosure include tobacco, potato, corn (maize), tomato, rice, wheat, alfalfa, soybean, and the like.

The phrase “transformed plant” refers to a plant comprising a nucleic acid construct, interchangeably termed a transgenic plant. The construct can be integrated into the plant's genome (i.e., nuclear or plastid genome), in some or at least substantially all of the cells of the plant. For example, the construct can be present in the plant's germline. Accordingly, the construct can be heritable, that is, inherited by at least one or more members, or at least substantially all members, of a succeeding generation of the plant.

The term “nucleic acid” refers to a compound comprising a chain of nucleotides. A nucleic acid can be single-stranded or double stranded. A nucleic acid can have a natural or artificial (i.e., engineered) structure, or a combination thereof. A nucleic acid can refer to ribonucleic acids, deoxyribonucleic acids, or a hybrid.

The term “gene” refers to a nucleic acid or segment thereof that provides an expressible unit for expression of a polypeptide and/or a functional RNA (e.g., an interfering RNA). A gene thus can include a targeting region (also termed a targeting sequence) to define the sequence of the interfering RNA that is expressed and at least one transcriptional promoter (also termed a promoter sequence) operatively linked to the targeting region, to control (i.e., promote, drive, and/or regulate) transcription of the targeting region. A gene optionally can include one or more other control regions and/or untranslated regions, such as at least one 5′ leader sequence, intron, transcriptional terminator (also termed a terminator sequence), or any combination thereof, among others.

The term “genetics” may refer to a subset or portion of the genome of a subject, which can be one or two nucleotides, a SNP, or a defined sequence, and determining the genetics of a subject may comprise, but is not limited to, identifying, locating, sequencing, probing, hybridizing to, quantifying or labeling one or more nucleic acid bases of the genome of the subject, which for example a subject may be a plant, seed or embryo or parts thereof.

The term “construct” refers to a nucleic acid created, at least in part, outside of plants using techniques of genetic engineering. A gene included in a construct can be termed a transgene.

The term “expression” refers to a process by which a product, namely, an RNA and/or a polypeptide, is synthesized based on information encoded in a nucleic acid and/or gene, generally in the form of DNA (or RNA). Accordingly, the nucleic acid/gene can be expressed to form an RNA and/or polypeptide, which means that the RNA and/or polypeptide is expressed from the nucleic acid/gene.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

### B. METHODS OF OBTAINING SEEDS COMPRISING CLONAL EMBRYOS

Disclosed are methods of obtaining seeds comprising clonal embryos comprising collecting one or more seeds produced by a maternal plant, wherein the maternal plant is unable to be pollinated, wherein the one or more seeds comprise a parthenogenically-derived embryo that is a clone of the maternal plant.

Disclosed are methods of obtaining seeds comprising clonal embryos comprising collecting one or more seeds produced by a maternal plant, wherein the maternal plant is unable to be pollinated, wherein the one or more seeds comprise a parthenogenically-derived embryo that is a clone of the maternal plant, wherein the maternal plant is defective in at least one RNA dependent DNA methylation pathway gene. For example, the RNA dependent DNA methylation pathway gene can be, but is not limited to, AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NRPD1b (NUCLEAR POLYMERASE D 1b), NRPD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9). Defective RNA-dependent DNA methylation can result in plants that are unable to form viable pollen.

In some instances, the AGO4 allele can be ago4-6 or ago4-1; the AGO6 allele can be ago6-2; the AGO9 allele can be 9-2, 9-3 or 9-4; the AGO8 allele can be ago 8-1; the RDR2 allele can be rdr2-1; the RDR6 allele can be rdr6-15 or rdr6-11; the SGS3 allele can be sgs3-11; the DRM2 allele can be drm2-2; and the MET1 allele can be met1-7.

Also disclosed are methods of obtaining seeds comprising clonal embryos comprising a) collecting one or more seeds produced by a maternal plant, wherein the maternal plant is defective in at least one RNA dependent DNA methylation pathway gene; and b) sorting the seeds to separate the seeds comprising clonal embryos from the seeds comprising non-clonal embryos; wherein the maternal plant is pollinated prior to collecting the seeds, and wherein the one or more seeds produced by the maternal plant comprise an embryo that is a clone of the maternal plant.

Sorting seeds can be based on phenotype or genotype. Sorting by phenotype can comprise sorting the seeds based on size, shape, color, or a combination thereof. Sorting can be performed manually or automatically. Automatic sorting can comprise a machine comprising an optical detector. In some instances, the sorting can be done visually.

The disclosed methods of obtaining seeds comprising clonal embryos can comprise a maternal plant that is unable to self-pollinate. A maternal plant's ability to self-pollinate can be disrupted physically, chemically, or genetically. Examples of chemical disruption comprise exposure to a gametocide that abolishes pollen formation. Gametocides can include, but are not limited to, at least one of maleic hydrazide (1,2-dihydropyridazine, 3-6-dione) (MH), 2,4-dichlorophenoxyacetic acid (2,4-D), a-naphthalene acetic acid (NAA), and tri-iodobenzoic acid (TIBA). Examples of physical disruption comprise emasculating the maternal plant. Emasculating occurs prior to collecting one or more seeds.

Also disclosed are methods of obtaining clonal seeds, the method comprising: (A) obtaining one or more maternal plants, each optionally being defective for RNA-dependent DNA methylation; (B) allowing pollination of the maternal plants; (C) collecting a mixture of seeds produced by the maternal plants and including seeds comprising non-clonal embryos that were sexually-generated and seeds comprising clonal embryos that are each a clone of a maternal plant; and (D) sorting the mixture to separate seeds comprising clonal embryos from seeds comprising non-clonal embryos by distinguishing clonal embryos from sexually-generated embryos.

Disclosed are methods of obtaining clonal seeds, the method comprising: (A) obtaining one or more maternal plants, each optionally being defective for RNA-dependent DNA methylation; (B) allowing pollination of the maternal plants; (C) collecting a mixture of seeds produced by the maternal plants and including seeds comprising non-clonal embryos that were sexually-generated and seeds comprising clonal embryos that are each a clone of a maternal plant; and (D) sorting the mixture to separate seeds comprising clonal embryos from seeds comprising non-clonal embryos by distinguishing clonal embryos from sexually-generated embryos, wherein the step of sorting includes a step of optically distinguishing clonal embryos from sexually-generated embryos.

Clonal embryos can be distinguished from sexually-generated embryos at least in part by color. In some instances, clonal embryos can be distinguished from sexually-generated embryos at least in part by size, shape, size and shape, or genetic testing.

Disclosed are methods of obtaining clonal seeds, the method comprising: (A) obtaining one or more maternal plants, each optionally being defective for RNA-dependent DNA methylation; (B) allowing pollination of the maternal plants; (C) collecting a mixture of seeds produced by the maternal plants and including non-clonal seeds containing sexually-generated embryos and clonal seeds containing clonal embryos that are each a clone of a maternal plant; and (D) sorting the mixture to separate clonal seeds from non-clonal seeds by distinguishing clonal embryos from sexually-generated embryos, wherein the step of sorting includes a step of optically distinguishing clonal embryos from sexually-generated embryos, wherein the step of sorting can be performed manually. In some instances, the step of sorting can be performed automatically. In some instances, the step of sorting can be performed with a machine including an optical detector.

Disclosed are methods of obtaining clonal seeds, the method comprising: (A) obtaining one or more maternal plants, each optionally being defective for RNA-dependent DNA methylation; (B) allowing pollination of the maternal plants; (C) collecting a mixture of seeds produced by the maternal plants and including non-clonal seeds containing sexually-generated embryos and clonal seeds containing clonal embryos that are each a clone of a maternal plant; and (D) sorting the mixture to separate clonal seeds from non-clonal seeds by distinguishing clonal embryos from sexually-generated embryos, wherein each maternal plant has at least one mutation that renders the maternal plant defective for RNA-dependent DNA methylation. A mutation that renders the maternal plant defective for RNA-dependent DNA methylation can modify at least one endogenous gene that encodes a product involved in and/or required for RNA-dependent DNA methylation. The mutation can include a mutation that occurred spontaneously or that was produced with a nonspecific chemical mutagen, a transposable element, or a targeting construct (e.g., CRE/LOX).

In any of the disclosed methods, each maternal plant can include at least one construct that renders the maternal plant defective for RNA-dependent DNA methylation. The construct can be any of the constructs disclosed herein. For example, the construct can comprise a siRNA that silences expression of a gene in the RNA-dependent DNA methylation pathway. In some instances, the at least one construct expresses at least one RNA that renders the maternal plant defective for RNA-dependent DNA methylation. RNA can include an RNA having a pair of regions configured to base-pair intramolecularly.

The step of obtaining one or more maternal plants can include a step of transforming an ancestor of the one or more maternal plants with at least one construct including an embryo marker and/or configured to affect a characteristic of an embryo marker provided by each maternal plant. A further step of transforming an ancestor of the one or more maternal plants with at least one construct to render RNA-dependent DNA methylation defective can be employed. A same ancestor of the one or more maternal plants can be transformed to introduce the embryo marker and render RNA-dependent DNA methylation defective. For example, the ancestor is transformed with a single construct including the embryo marker and configured to affect RNA-dependent DNA methylation.

Each maternal plant can include an embryo marker introduced by breeding in the disclosed methods of obtaining clonal seeds. The embryo marker can be an allelic variant, such as a mutant, of an endogenous gene.

Disclosed are methods of obtaining clonal seeds, wherein an embryo marker can be provided by pollen.

Disclosed are methods of obtaining clonal seeds, wherein none of the maternal plants contributes pollen for the step of allowing pollination.

Disclosed are methods of obtaining clonal seeds, wherein each of the maternal plants can be male sterile.

In some instances, each of the maternal plants can be exposed to a gametocide that abolishes pollen formation before the step of allowing pollination, and wherein the gametocide includes at least one of maleic hydrazide (1,2-dihydropyridazine, 3-6-dione) (MH), 2,4-dichlorophenoxyacetic acid (2,4-D), a-naphthalene acetic acid (NAA), and tri-iodobenzoic acid (TIBA).

In some instances the methods of obtaining clonal seeds further comprises a step of emasculating each of the maternal plants before the step of allowing pollination.

Disclosed are methods of obtaining clonal seeds, wherein each maternal plant has an abnormal level, activity, and/or structure of at least one of the following genes/gene products: AGO4 (ARGONAUTE 4), AGO6 (ARGONAUTE 6), AGO9 (ARGONAUTE 9), CMT3 (CHROMOMETHYLASE 3), DCL3 (DICER-LIKE 3), DRM2 (DOMAINS REARRANGED METHYLASE 2), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

Also disclosed are methods of obtaining clonal seeds, the method comprising: (A) obtaining one or more maternal plants each defective for RNA-dependent DNA methylation and each unable to form, or prevented from forming, viable pollen; and (B) collecting seeds produced by the one or more maternal plants, each seed containing an embryo that is a clone of the maternal plant. In some instances, each maternal plant is unable to form viable pollen.

Disclosed are methods of obtaining clonal seeds, the method comprising: (A) obtaining one or more maternal plants each defective for RNA-dependent DNA methylation and each unable to form, or prevented from forming, viable pollen; and (B) collecting seeds produced by the one or more maternal plants, each seed containing an embryo that is a clone of the maternal plant, wherein each maternal plant is unable to form viable pollen, wherein each maternal plant contains at least one construct that renders the maternal plant unable to form viable pollen.

Disclosed are methods of obtaining clonal seeds, the method comprising: (A) obtaining one or more maternal plants each defective for RNA-dependent DNA methylation and each unable to form, or prevented from forming, viable pollen; and (B) collecting seeds produced by the one or more maternal plants, each seed containing an embryo that is a clone of the maternal plant, wherein each maternal plant is unable to form viable pollen, wherein each maternal plant has one or more mutations that render the maternal plant unable to form viable pollen.

Disclosed are methods of obtaining clonal seeds, the method comprising: (A) obtaining one or more maternal plants each defective for RNA-dependent DNA methylation and each unable to form, or prevented from forming, viable pollen; and (B) collecting seeds produced by the one or more maternal plants, each seed containing an embryo that is a clone of the maternal plant, wherein the step of obtaining includes a step of emasculating each maternal plant by removing one or more male reproductive organs, namely, stamens, from the maternal plant.

Disclosed are methods of obtaining clonal seeds, the method comprising: (A) obtaining one or more maternal plants each defective for RNA-dependent DNA methylation and each unable to form, or prevented from forming, viable pollen; and (B) collecting seeds produced by the one or more maternal plants, each seed containing an embryo that is a clone of the maternal plant, wherein the step of obtaining includes a step of exposing each maternal plant to a substance (a gametocide) that abolishes formation of viable pollen.

### C. METHODS OF PLANT CLONING

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein each maternal plant includes at least one construct that renders the maternal plant defective for RNA-dependent DNA methylation.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein each maternal plant includes at least one construct that renders the maternal plant defective for RNA-dependent DNA methylation, wherein the at least one construct expresses at least one RNA that renders the maternal plant defective for RNA-dependent DNA methylation. For example, the at least one RNA can include an RNA having a pair of regions configured to base-pair intramolecularly.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein the step of obtaining one or more maternal plants includes a step of transforming an ancestor of the one or more maternal plants with at least one construct including an embryo marker and/or configured to affect a characteristic of an embryo marker provided by each maternal plant.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein the step of obtaining one or more maternal plants includes a step of transforming an ancestor of the one or more maternal plants with at least one construct including an embryo marker and/or configured to affect a characteristic of an embryo marker provided by each maternal plant, further comprising a step of transforming an ancestor of the one or more maternal plants with at least one construct to render RNA-dependent DNA methylation defective. In some instances, a same ancestor of the one or more maternal plants can be transformed to introduce the embryo marker and render RNA-dependent DNA methylation defective. In some instances, the ancestor can be transformed with a single construct including the embryo marker and configured to affect RNA-dependent DNA methylation.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein each maternal plant includes an embryo marker introduced by breeding. The embryo marker can be an allelic variant, such as a mutant, of an endogenous gene. An embryo marker can be provided by pollen.

Disclosed are methods of plant cloning, wherein none of the maternal plants contributes pollen for the step of allowing pollination.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein each of the maternal plants is male sterile.

In some instances, each of the maternal plants can be exposed to a gametocide that abolishes pollen formation before the step of allowing pollination, and wherein the gametocide includes at least one of maleic hydrazide (1,2-dihydropyridazine, 3-6-dione) (MH), 2,4-dichlorophenoxyacetic acid (2,4-D), a-naphthalene acetic acid (NAA), and tri-iodobenzoic acid (TIBA).

The disclosed methods of plant cloning can further comprise a step of emasculating each of the maternal plants before the step of allowing pollination.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein each maternal plant has an abnormal level, activity, and/or structure of at least one of the following genes/gene products: AGO4 (ARGONAUTE 4), AGO6 (ARGONAUTE 6), AGO9 (ARGONAUTE 9), CMT3 (CHROMOMETHYLASE 3), DCL3 (DICER-LIKE 3), DRM2 (DOMAINS REARRANGED METHYLASE 2), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein the step of growing progeny plants includes a step of growing seedlings, and wherein the step of distinguishing is performed while the progeny plants are seedlings. In some instances, the step of distinguishing can be performed by optically distinguishing clonal plants from sexually-generated plants.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, wherein clonal plants are visually distinguishable from sexually-generated plants. In some instances, the step of distinguishing can include a step of performing at least one test on the progeny plants. For example, the at least one test can include a genetic test such as, but not limited to, genetic profiling.

Disclosed are methods of plant cloning, the method comprising: (A) obtaining one or more maternal plants, each optionally being defective for RNA-dependent DNA methylation; (B) allowing pollination of the maternal plants; (C) growing progeny plants from seeds produced by the one or more maternal plants, the progeny plants including sexually-generated plants and clonal plants, each clonal plant being a clone of a maternal plant; and (D) distinguishing clonal plants from sexually-generated plants, further comprising a step of sorting clonal plants from sexually-generated plants.

### D. METHODS OF SCREENING

Disclosed are methods of screening for maternal plants that produce seeds comprising parthenogenically-derived clonal embryos comprising a) obtaining a maternal plant unable to pass on paternally-derived chromosomes to embryos, wherein an activity of a gene of interest in a RNA dependent DNA methylation pathway is silenced in the plant; b) harvesting the seeds; and c) determining whether the seeds comprise clonal embryos, wherein the presence of seeds comprising clonal embryos indicates the maternal plant can produce parthenogenically-derived clonal embryos.

Disclosed are methods of screening for maternal plants that produce seeds comprising parthenogenically-derived clonal embryos comprising a) obtaining a maternal plant unable to pass on paternally-derived chromosomes to embryos, wherein an activity of a gene of interest in a RNA dependent DNA methylation pathway is silenced in the plant; b) harvesting the seeds; and c) determining whether the seeds comprise clonal embryos, wherein the presence of seeds comprising clonal embryos indicates the maternal plant can produce parthenogenically-derived clonal embryos, wherein the activity of the gene of interest is silenced using RNA interference.

Disclosed are methods of screening for maternal plants that produce seeds comprising parthenogenically-derived clonal embryos comprising a) obtaining a maternal plant unable to pass on paternally-derived chromosomes to embryos, wherein an activity of a gene of interest in a RNA dependent DNA methylation pathway is silenced in the plant; b) harvesting the seeds; and c) determining whether the seeds comprise clonal embryos, wherein the presence of seeds comprising clonal embryos indicates the maternal plant can produce parthenogenically-derived clonal embryos, wherein the gene of interest is AGO4, AGO6, AGO8, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9.

### E. METHODS OF INCREASING YIELD OF CLONAL SEEDS

Disclosed are methods of increasing the yield of seeds comprising parthenogenically-derived clonal embryos comprising a) obtaining a maternal plant unable to pass on paternally-derived chromosomes to embryos, wherein an activity of a gene of interest in a RNA dependent DNA methylation pathway is silenced in the plant; b) pollinating the maternal plant; c) collecting seeds produced by the maternal plant; d) sorting the seeds comprising parthenogenically-derived clonal embryos from seeds comprising non-clonal embryos.

Sorting seeds comprising parthenogenically-derived clonal embryos from seeds comprising non-clonal embryos can be based on phenotype or genotype. Sorting based on phenotype comprises determining the size, shape, color, or a combination thereof, of the seeds. Sorting can be performed manually or automatically. Automatic sorting can comprise a machine comprising an optical detector. In some instances, sorting can be done visually

Disclosed are methods of increasing the yield of seeds comprising parthenogenically-derived clonal embryos comprising a) obtaining a maternal plant unable to pass on paternally-derived chromosomes to embryos, wherein an activity of a gene of interest in a RNA dependent DNA methylation pathway is silenced in the plant; b) pollinating the maternal plant; c) collecting seeds produced by the maternal plant; d) sorting the seeds comprising parthenogenically-derived clonal embryos from seeds comprising non-clonal embryos, wherein the RNA dependent DNA methylation pathway gene is AGO4, AGO6, AGO8, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9.

### F. CONSTRUCTS

Disclosed are constructs comprising a nucleic acid sequence. The nucleic acid sequence can render the maternal plant defective for RNA-dependent DNA methylation. The nucleic acid sequence can silence activity of a RNA-dependent DNA methylation pathway gene. The nucleic acid sequence can be exogenous to plant sequences.

Disclosed are construct comprising a nucleic acid sequence that renders the maternal plant defective for RNA-dependent DNA methylation, and wherein the maternal plant produces seeds comprising parthenogenically-derived clonal embryos.

Disclosed are constructs comprising a nucleic acid sequence that renders the maternal plant defective for RNA-dependent DNA methylation, wherein the RNA dependent DNA methylation pathway gene is AGO4), AGO6, AGOG, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9.

The constructs can comprise a vector backbone of any known vector used to deliver nucleic acids to plants. For example the constructs can be plasmids or nanoparticles. These constructs can be used in methods for producing transgenic plants which are well known to those skilled in the art. Transgenic plants can be produced by a variety of different transformation methods including, but limited to, microinjection; electroporation; microprojectile bombardment, also known as particle acceleration or biolistic bombardment; viral mediated transformation or Agrobacterium-mediated transformation (see for example U.S. Pat. Nos. 5,405,765; 5,472,869; 5,538,877; 5,538,880; 5,550,318; 5,641,664; 5,736,369; Watson et al. Recombinant DNA, Scientific American Books 1992). An example of a commonly used plasmid is pBIN19 (Lee and Gelvin, 2008; Plant Physiology 146:235-332). pBIN19 carries two antibiotic resistance genes, one on the plasmid with a bacterial promoter to allow for selection of bacteria that have the plasmid. A second one is included within the T-DNA region driven by a plant promoter to allow for selection of transformed plant cells. Other examples of commonly used plasmids include pBI101 (Genbank Accession AAC53706) and pBI121 Genbank Accession AF485783), pMDC100 (TAIR Accession 1009003749), and pFGC5941 (Genbank Accession AY310901).

Plant transformation can be accomplished by several methods. DNA can be introduced in single cells and thereafter regenerated into complete plants by tissue culture. Other transformation methods can only be applied to protoplasts (cells from which the walls have been removed). Particle bombardment and the natural vector Agrobacterium tumefaciens can be used as they rely on whole plant tissues such as roots and leaves, which are easier to handle and require less of the lengthy steps that are required for plant regeneration. In some species, Agrobacterium transformation can also be used by infiltrating or dipping intact flower buds. Several techniques for direct DNA delivery can be used, such as but not limited to, uptake of DNA into isolated protoplasts mediated by chemical procedures, electroporation, and injection and the use of high-velocity particles to introduce DNA into intact tissues. Direct DNA uptake is applicable to both stable and transient gene expression studies and relies on a range of vectors, including those employed for gene cloning. Although the frequency of stable transformation can be low, direct DNA uptake is applicable to those plants not amenable to Agrobacterium transformation, particularly monocotyledons. Bacteria and plant tissues are cultured together to allow transfer of foreign DNA into plant cells then transformed plants are regenerated on selection media. Any number of different organs and tissues can serve as targets from Agrobacterium mediated transformation as described specifically for members of the Brassicaceae. These include thin cell layers (Charest, P. J., et al, 1988, Theor. Appl. Genet. 75:438-444), hypocotyls (DeBlock, M., et al, 1989, Plant Physiol. 91:694-701), leaf discs (Feldman, K. A., and Marks, M. D., 1986, Plant Sci. 47:63-69), stems (Fry J., et al, 1987, Plant Cell Repts. 6:321-325), cotyledons (Moloney M. M., et al, 1989, Plant Cell Repts. 8:238-242) and embryoids (Neuhaus, G., et al, 1987, Theor. Appl. Genet. 75:30-36), or even whole plants using in vacuum infiltration and floral dip or floral spraying transformation procedures available in Arabidopsis and Medicago at present but likely applicable to other plants in the hear future. It is understood, however, that it may be desirable in some crops to choose a different tissue or method of transformation.

Other methods that have been employed for introducing recombinant molecules into plant cells involve mechanical means such as direct DNA uptake, liposomes, electroporation (Guerche, P. et al, 1987, Plant Science 52:111-116) and micro-injection (Neuhaus, G., et al, 1987, Theor. Appl. Genet. 75:30-36). The possibility of using microprojectiles and a gun or other device to force small metal particles coated with DNA into cells has also received considerable attention (Klein, T. M. et al., 1987, Nature 327:70-73).

It is often desirable to have the DNA sequence in homozygous state which may require more than one transformation event to create a parental line, requiring transformation with a first and second recombinant DNA molecule both of which encode the same gene product. It is further contemplated in some of the embodiments of the process of the invention that a plant cell be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it may be possible to perform a sexual cross between individual plants or plant lines containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques and/or Southern blot techniques or PCR-based methods known to those of skill in the art.

A large number of plants have been shown capable of regeneration from transformed individual cells to obtain transgenic whole plants. For example, regeneration has been shown for dicots as follows: apple, Malus pumila (James et al., Plant Cell Reports (1989) 7:658); blackberry, Rubus, Blackberry/raspberry hybrid, Rubus, red raspberry, Rubus (Graham et al., Plant Cell, Tissue and Organ Culture (1990) 20:35); carrot, Daucus carota (Thomas et al., Plant Cell Reports (1989) 8:354; Wurtele and Bulka, Plant Science (1989) 61:253); cauliflower, Brassica oleracea (Srivastava et al., Plant Cell Reports (1988) 7:504); celery, Apium graveolens (Catlin et al., Plant Cell Reports (1988) 7:100); cucumber, Cucumis sativus (Trulson et al., Theor. Appl. Genet. (1986) 73:11); eggplant, Solanum melonoena (Guri and Sink, J. Plant Physiol. (1988) 133:52) lettuce, Lactuca sativa (Michelmore et al., Plant Cell Reports (1987) 6:439); potato, Solanum tuberosum (Sheerman and Bevan, Plant Cell Reports (1988) 7:13); rape, Brassica napus (Radke et al., Theor. Appl. Genet. (1988) 75:685; Moloney et al., Plant Cell Reports (1989) 8:238); soybean (wild), Glycine canescens (Rech et al., Plant Cell Reports (1989) 8:33); strawberry, Fragaria × ananassa (Nehra et al., Plant Cell Reports (1990) 9:10; tomato, Lycopersicon esculentum (McCormick et al., Plant Cell Reports (1986) 5:81); walnut, Juglans regia (McGranahan et al., Plant Cell Reports (1990) 8:512); melon, Cucumis melo (Fang et al., 86th Annual Meeting of the American Society for Horticultural Science Hort. Science (1989) 24:89); grape, Vitis vinifera (Colby et al., Symposium on Plant Gene Transfer, UCLA Symposia on Molecular and Cellular Biology J Cell Biochem Suppl (1989) 13D:255; mango, Mangifera indica (Mathews, et al., symposium on Plant Gene Transfer, UCLA Symposia on Molecular and Cellular Biology J Cell Biochem Suppl (1989) 13D:264); and for the following monocots: rice, Oryza sativa (Shimamoto et al., Nature (1989) 338:274); rye, Secale cereale (de la Pena et al., Nature (1987) 325:274); maize, (Rhodes et al., Science (1988) 240:204).

Examples of vectors are pFGC5941 (Accession AY310901), pRS300 (Addgene plasmid 22846; Schwab et al. Plant Cell. 2006 May, 18(5):1121-33), pHELLSGATE (Accession AJ311874), and pMDC32 (Accession FJ172534.1) as listed here in Table 1.

TABLE 1

| SEQ | POLYNUCLEOTIDE/ | |
|---|---|---|
| ID NO: | POLYPEPTIDE | IDENTITY |
| 1 | POLYNUCLEOTIDE | pFGC5941 GenBank: AY310901.1 |
| 2 | POLYNUCLEOTIDE | pRS300 |
| | | Addgene plasmid 22846 |
| | | Article: Highly specific gene silencing |
| | | by artificial microRNAs in Arabidopsis. |
| | | Schwab et al (Plant Cell. 2006 May. |
| | | 18(5): 1121-33. PubMed) |
| 3 | POLYNUCLEOTIDE | pHELLSGATE GenBank: AJ311874.1 |
| 4 | POLYNUCLEOTIDE | pMDC32 GenBank: FJ172534.1 |

### G. MATERNAL PLANTS

Disclosed are maternal plants comprising any of the constructs described herein.

Disclosed are maternal plants comprising a construct, wherein the construct comprises a nucleic acid sequence that renders the maternal plant defective for RNA-dependent DNA methylation, and wherein the maternal plant produces seeds comprising parthenogenically-derived clonal embryos

Disclosed are maternal plants comprising a construct, wherein the construct comprises a nucleic acid sequence that renders the maternal plant defective for RNA-dependent DNA methylation, wherein the RNA dependent DNA methylation pathway gene is AGO4), AGO6, AGOG, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9.

Disclosed are maternal plants comprising a construct, wherein the construct comprises an exogenous nucleic acid sequence, wherein the construct renders the maternal plant defective for RNA-dependent DNA methylation. The exogenous nucleic acid sequence can silence activity of a RNA-dependent DNA methylation pathway gene.

Disclosed are maternal plants comprising a construct, wherein the construct comprises an exogenous nucleic acid sequence, wherein the construct renders the maternal plant defective for RNA-dependent DNA methylation, wherein the maternal plant further comprises a clonal seed.

Also disclosed are maternal plants comprising a defective RNA-dependent DNA methylation pathway gene. RNA dependent DNA methylation pathway genes that can be defective can be, but are not limited to, AGO4, AGO6, AGOG, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9.

Disclosed are maternal plants for production of seeds comprising clonal embryos, the maternal plant being defective for RNA-dependent DNA methylation and, when pollinated, producing seeds comprising sexually-generated embryos and seeds comprising clonal embryos that are each a clone of the maternal plant, the clonal embryos being optically distinguishable from the sexually-generated embryos.

Disclosed are maternal plants for production of clonal seeds, the maternal plant being defective for RNA-dependent DNA methylation and, when pollinated, producing seeds that form sexually-generated progeny plants and clonal progeny plants, with each clonal progeny plant being a clone of the maternal plant, the sexually-generated progeny plants being optically distinguishable from the sexually-generated progeny plants.

Disclosed are maternal plants for production of clonal seeds, the maternal plant being defective for RNA-dependent DNA methylation and unable to form viable pollen.

Disclosed are maternal plants for production of clonal seeds, the maternal plant being defective for RNA-dependent DNA methylation and treated to prevent formation of viable pollen.

### H. SEEDS

Disclosed are seeds comprising a parthenogenically-derived clonal embryo comprising a defective RNA-dependent DNA methylation pathway gene.

Disclosed are seeds comprising a parthenogenically-derived clonal embryo comprising a defective RNA-dependent DNA methylation pathway gene, wherein the seed, when grown, produces seeds comprising clonal embryos. Disclosed are seeds comprising a parthenogenically-derived clonal embryo comprising a defective RNA-dependent DNA methylation pathway gene.

Disclosed are seeds comprising a parthenogenically-derived clonal embryo comprising a defective RNA-dependent DNA methylation pathway gene, wherein the RNA dependent DNA methylation pathway gene is AGO4), AGO6, AGOG, AGO9, CMT3, DCL3, DRM2, EXS1, IDN2, MET1, NPRD1a, NPRD1b, NPRD2, NRPE1, NRPE2, RDR2, RDR6, SGS3, SUVH2, and SUVH9

### EXAMPLES

The following examples describe selected aspects and embodiments of the present disclosure, such as exemplary methods of generating and using maternal plants that produce clonal seeds, methods of distinguishing clonal seeds/progeny from sexually-generated seeds/progeny, and methods of obtaining clonal seeds/progeny from a mixture/set of clonal seeds/progeny and sexually-generated seeds/progeny, among others. The examples are presented for illustration only and are not intended to define or limit the scope of the present disclosure.

### I. EXAMPLE 1

### Single Gene Mutations Causing Self-Propagated Asexual Reproduction Through Seeds (Apomixis) in Arabidopsis

This example presents experimental results demonstrating the ability to clone a maternal plant through seeds.

1. Results

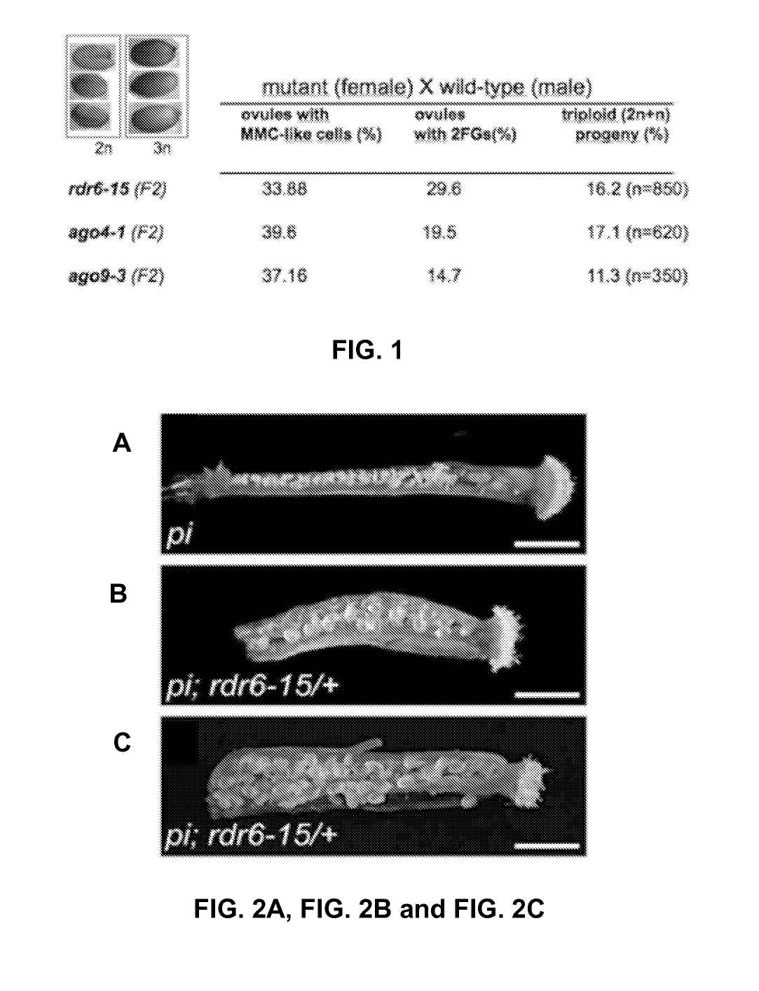

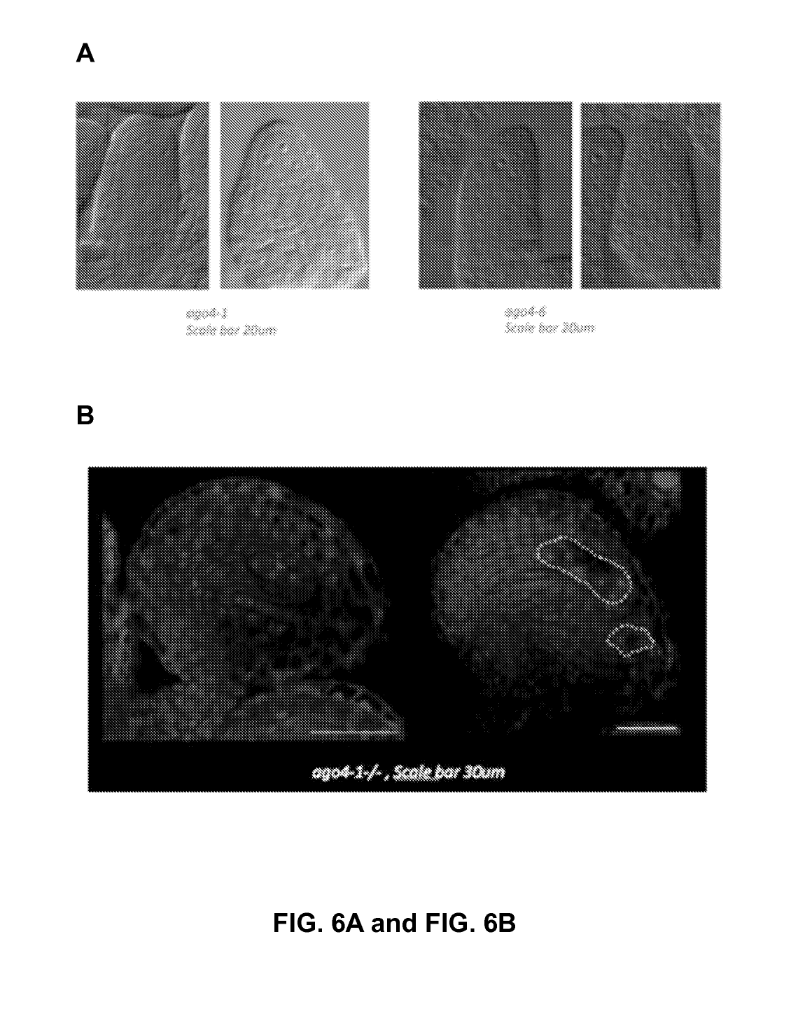

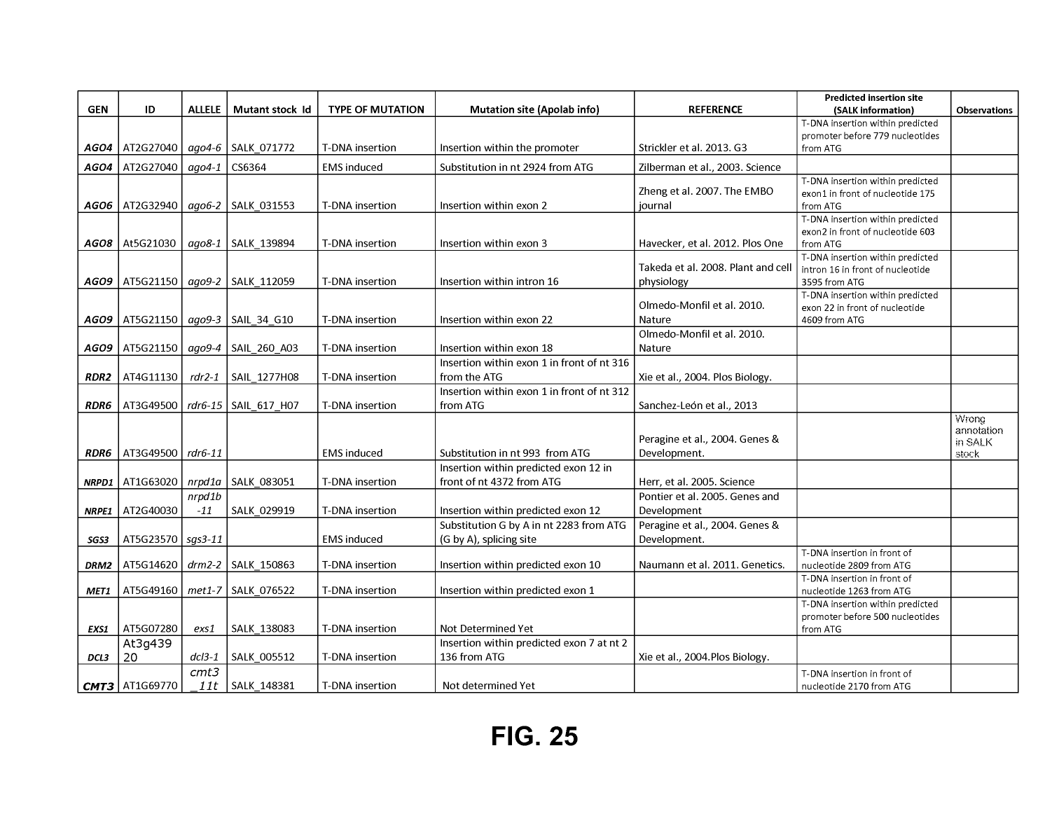

Previous results showed that dominant mutations in ARGONAUTE9 (AGO9) and RNA-DEPENDENT RNA POLYMERASE6 (RDR6) lead to differentiation of multiple female gametic cells that are able to initiate gametogenesis without undergoing meiosis by a mechanism reminiscent of apospory, a specific type of apomixis (Olmedo-Monfil et al., 2010; Duran-Figueroa and Vielle-Calzada, 2010). This phenotype was also found in mutants of the RNA-dependent DNA methylation (RdDM) pathway such as RNA-DEPENDENT RNA POLYMERASE2 (RDR2) and DICER-LIKE3 (DCL3). Mutations in the 24-nucleotide small RNA binding protein ARGONAUTE4 (AGO4), that is also a key member of the RdDM pathway required for de novo DNA methylation of heterochromatic regions (Law and Jacobsen, 2010), show an equivalent phenotype. Plants heterozygous for ago4-1 or ago4-6 were fertile and did not show signs of seed abortion; however, they showed pre-meiotic ovules with enlarged sub-epidermal cells containing a conspicuous nucleus, and at later developmental stages, several developing female gametophytes ectopically growing at both the micropylar and chalazal regions of the ovule (FIG. 1 and FIG. 6). A complete list of mutants identified as showing these ectopic female gametophytes reminiscent of apospory is provided in FIG. 25. To determine if defects in these mutants could lead to the formation of viable diploid female gametes, heterozygous rdr6-15, ago4-1, and agog-3 individuals were crossed to wild-type pollen and ploidy levels in the resulting progeny were determined. For all three mutants crossed as a female parent, triploid individuals were recovered at frequencies ranging between 11.3 and 17.1% (FIG. 1), indicating that plants defective in RDR6, AGO4 and AGO9 can form triploid embryos resulting from the fertilization of a diploid female gamete by a haploid sperm cell.

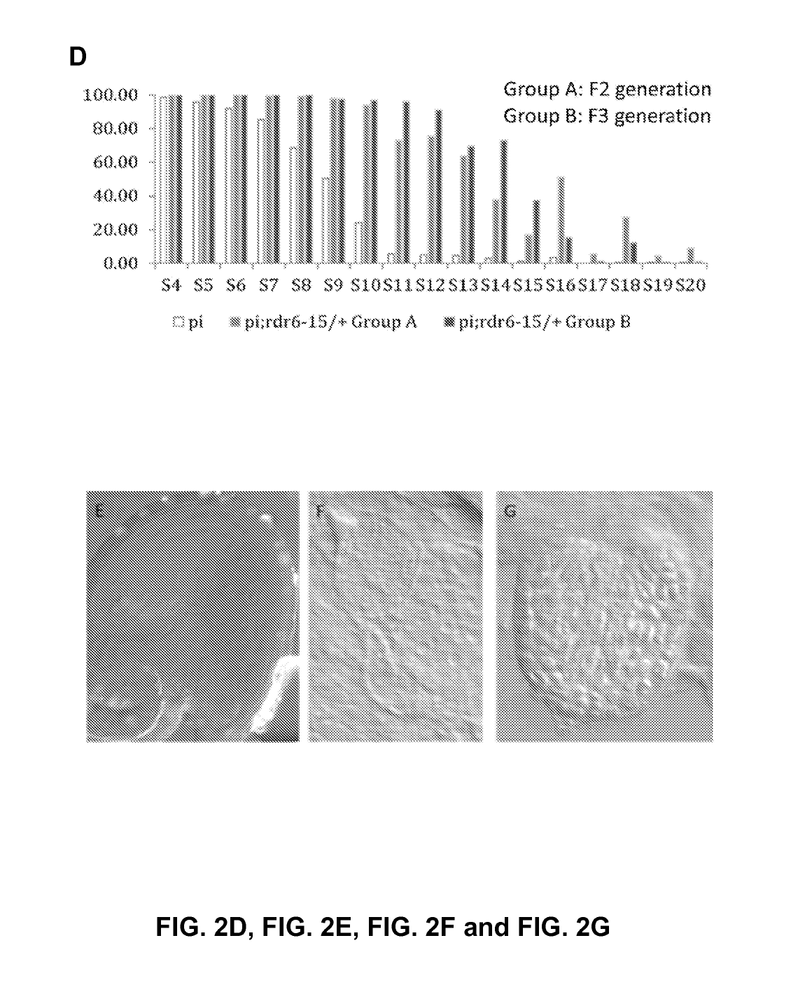

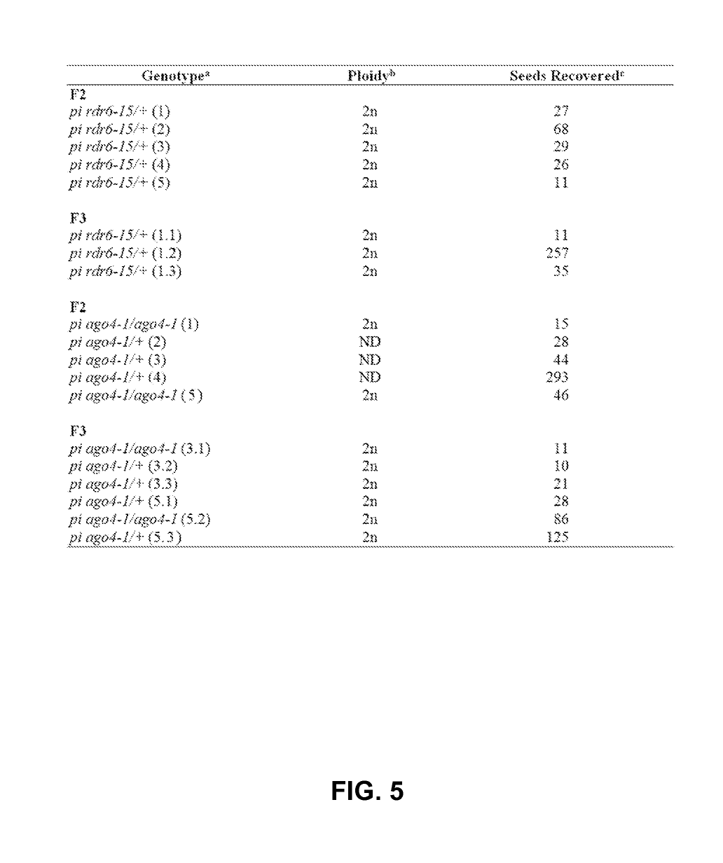

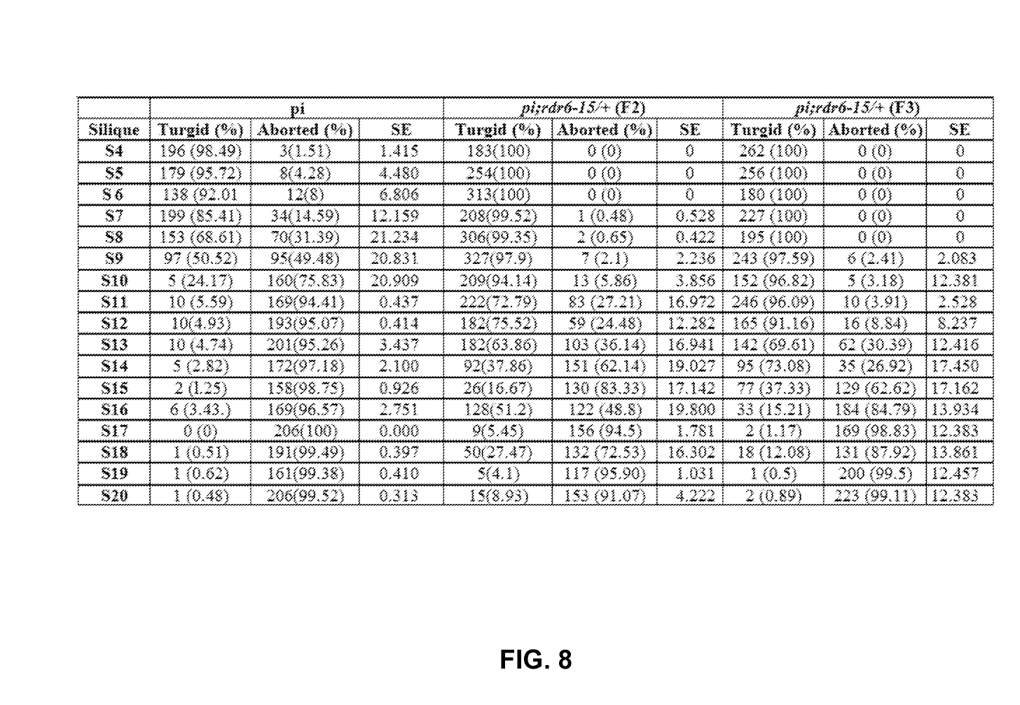

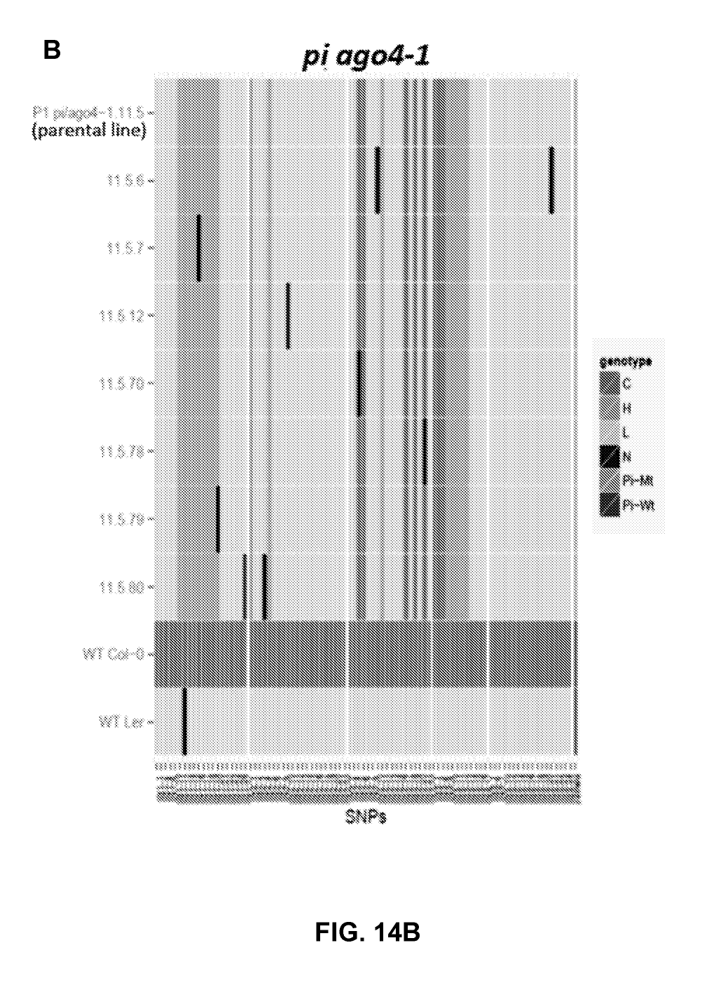

To determine if female gametes in some of these mutants are capable of initiating autonomous development of either an embryo or the endosperm, fully male sterile pistillata (pi) plants that completely lack stamens were crossed to homozygous ago4-1 or rdr6-15 individuals, and generated a corresponding F2 population. Previous reports showed that fertilized ovules of pi individuals develop into seeds, but unfertilized ovules remain small and eventually shrivel (Chaudhury et al., 1997). To avoid any cross-pollination, each F2 individual was genotyped before flowering and 79 pi rdr6-15/+, and 122 pi ago4-1/+ or pi ago4-1/ago4-1 diploid plants that were grown to maturity in full isolation from any source of pollen were identified. In contrast to ago4 for which homozygous pi ago4-1 individuals could be recovered, all rdr6-15 F2 segregants showing the pi phenotype were heterozygous for the rdr6-15 allele, a result indicating a male gametophytic lethal effect or a prevalence of clonal seeds. Because the elongation of individual siliques—presumed to be indicative of seed initiation (Ohad et al., 1996; Chadhury et al., 1997)—was not observed in any male sterile individual showing the pi phenotype, each consecutive developing silique was manually dissected from a group of selected pi rdr6-15/+ stems, scoring the number of developing seeds that showed significant enlargement and prevalent turgidity when compared to unfertilized ovules, a morphology comparable to the seeds developing after pollination of the pi plants. No reversion of the pi phenotype was observed in any of the individuals grown to maturity. Strikingly, pi rdr6-15/+ siliques showed an unusual frequency of developing seeds at stages when most ovules present in unpollinated gynoecia of pi control plants have collapsed (FIG. 2A to 2D and FIG. 8). Most of these developing seeds grow and persist for several days without showing dehydration or shriveling. The phenotype was consistently observed in individuals from both F2 and F3 generations. A detailed observation of whole-mount cleared developing seeds showed the presence of free nuclei in the central cell and a developing embryo at the micropylar region, confirming that these plants do initiate the formation of both an embryo and its companion endosperm (FIGS. 2E and 2G). To determine if some of these developing seeds reach maturity, ten diploid F2 and ten F3 individuals showing the pi phenotype and carrying mutations in either rdr6-15 or ago4-1 were systematically harvested and searched for phenotypic evidence of seed formation by manually dissecting each independent silique at maturity. Strikingly, whereas progeny was completely absent in pi controls, all individuals carrying rdr6-15 or ago4-1 mutations produced a variable number of phenotypically normal mature seeds. For pi rdr6-15/+ individuals, the number of seeds per plant ranged between 11 and 125, whereas pi ago4-1/+ and pi ago4-1/ago4-1 plants produced between 10 and 293 seeds per plant, with no clear quantitative distinction between both genotypes or a specific generation (FIG. 5 and FIGS. 13A and 13B).

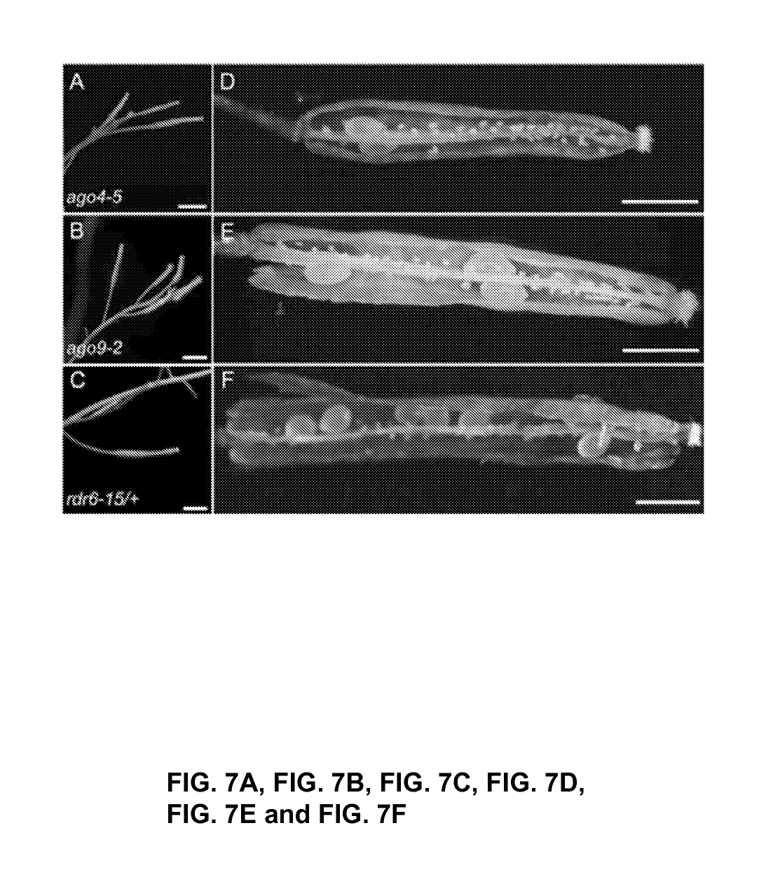

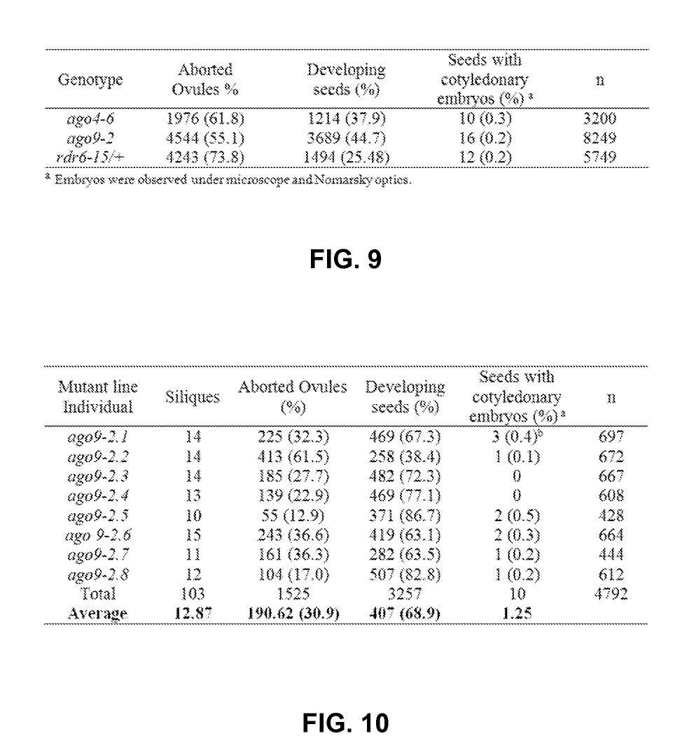

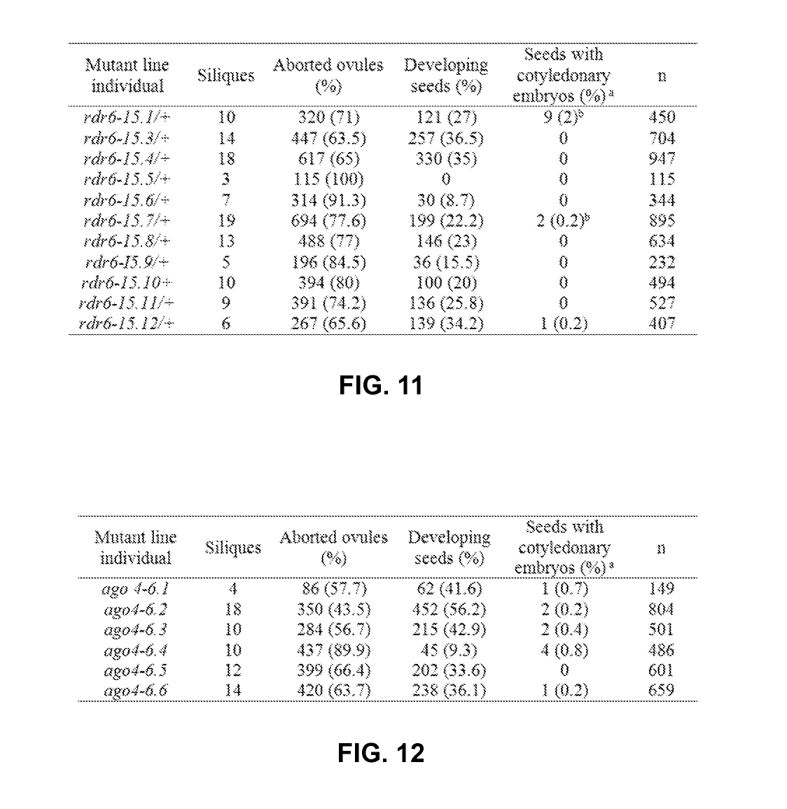

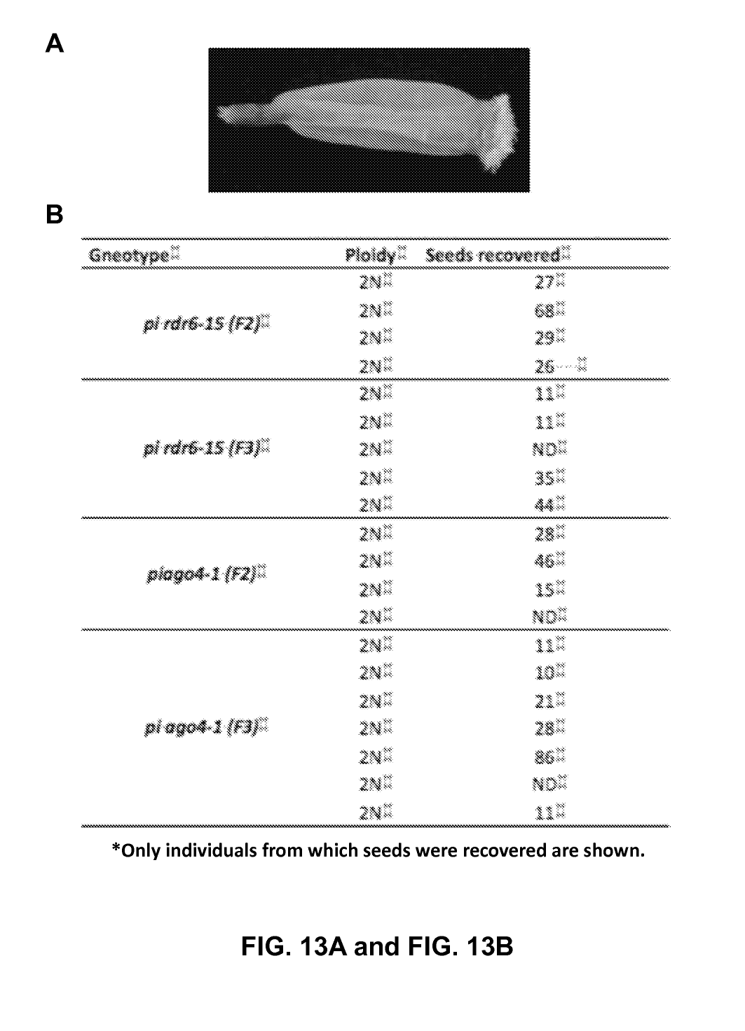

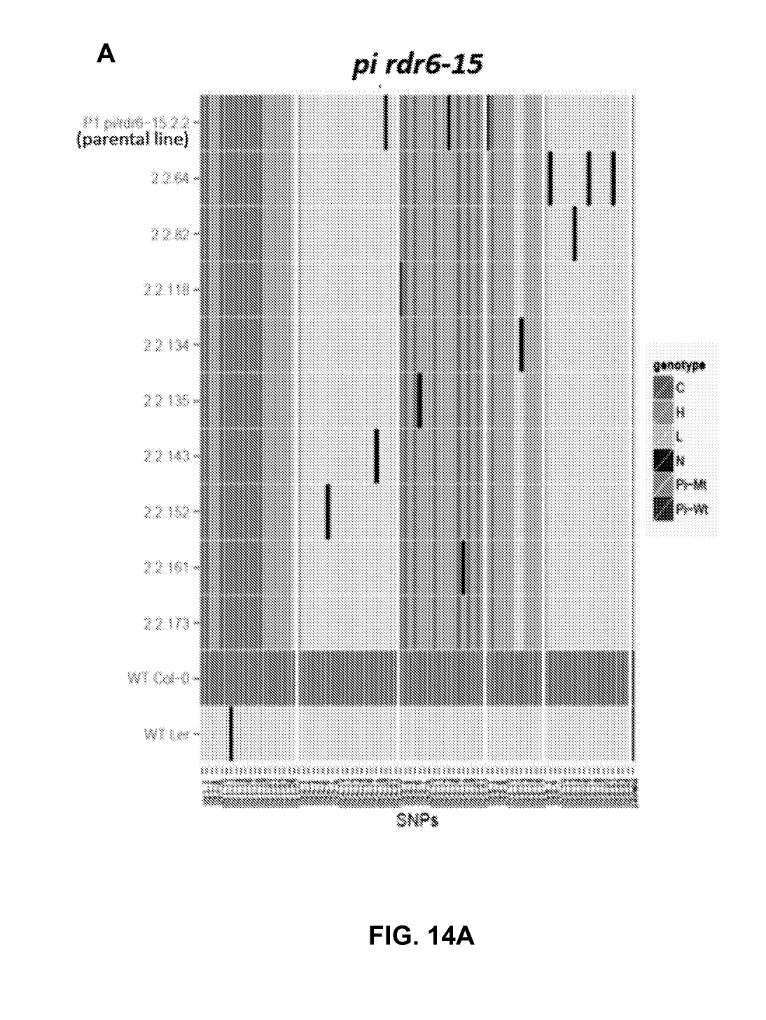

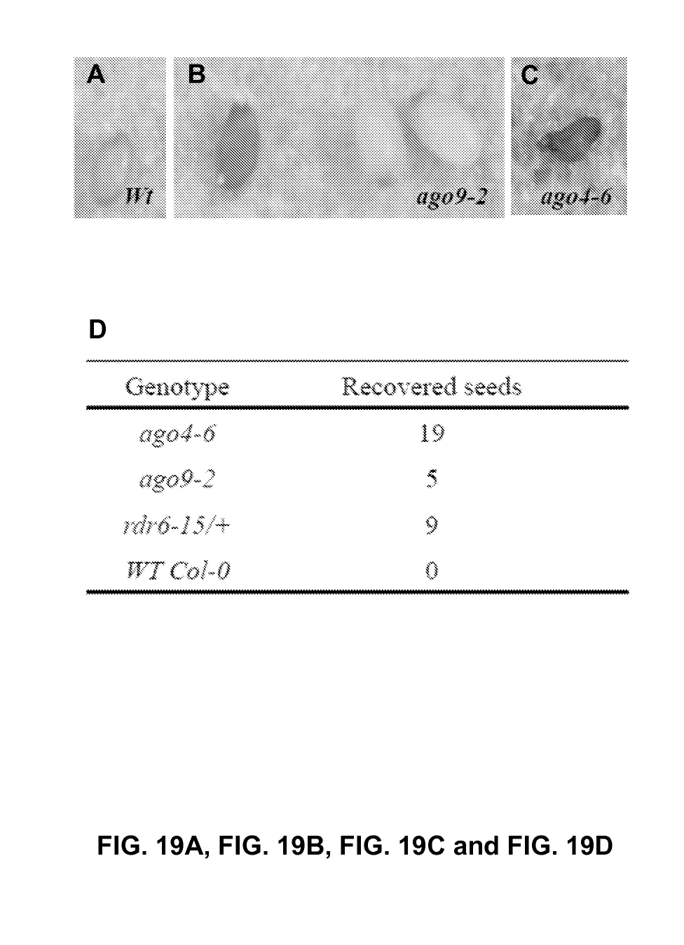

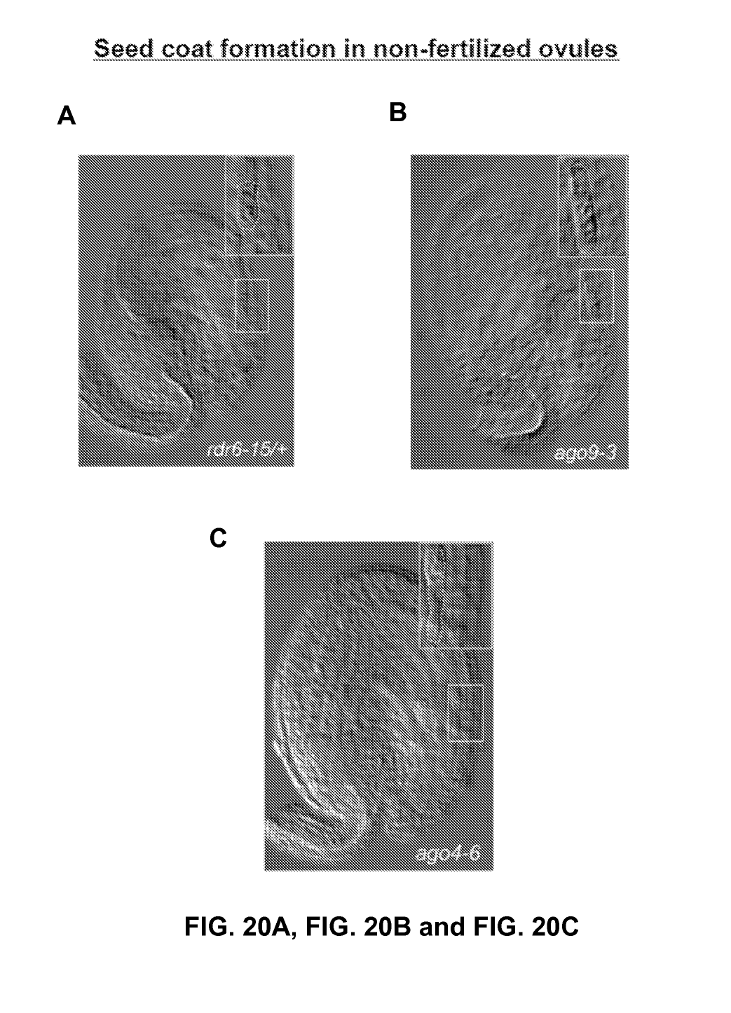

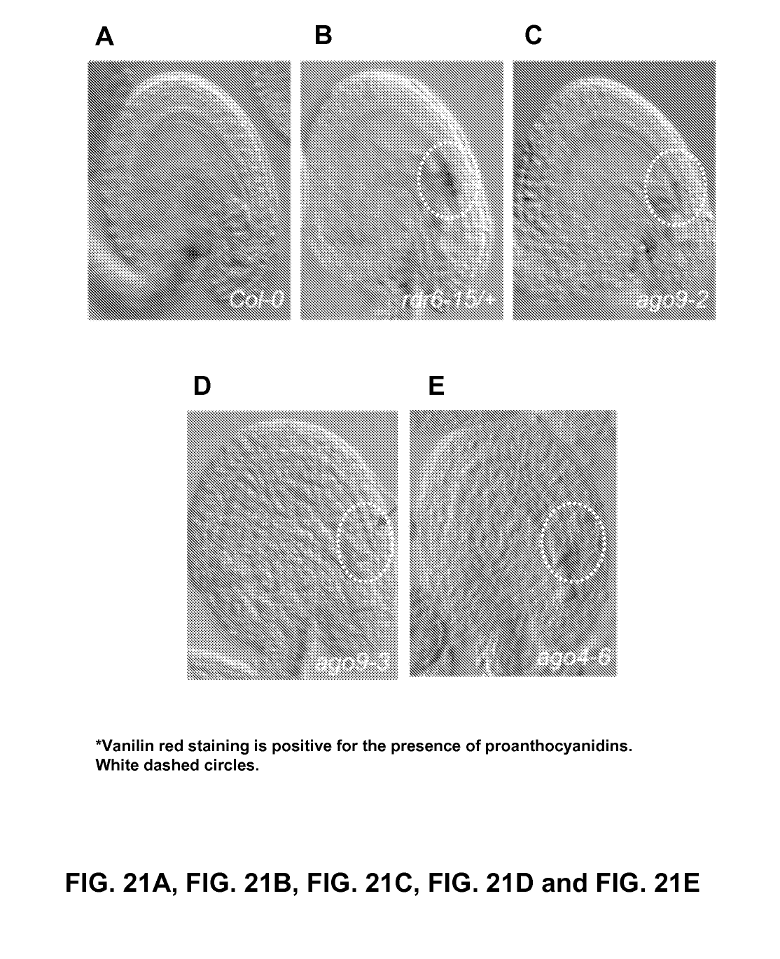

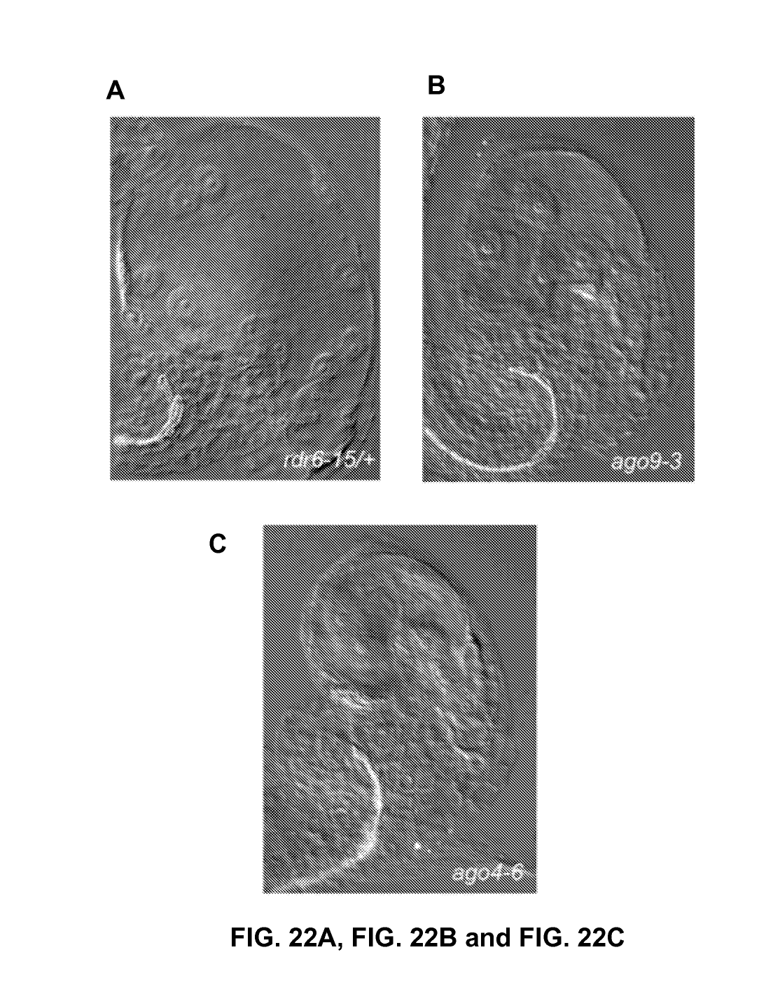

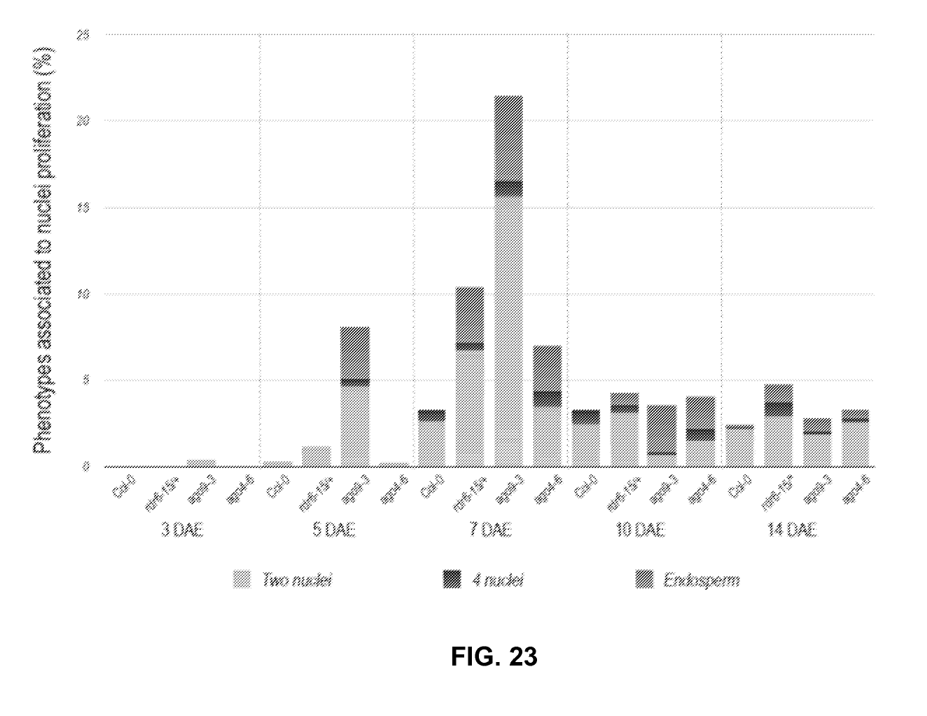

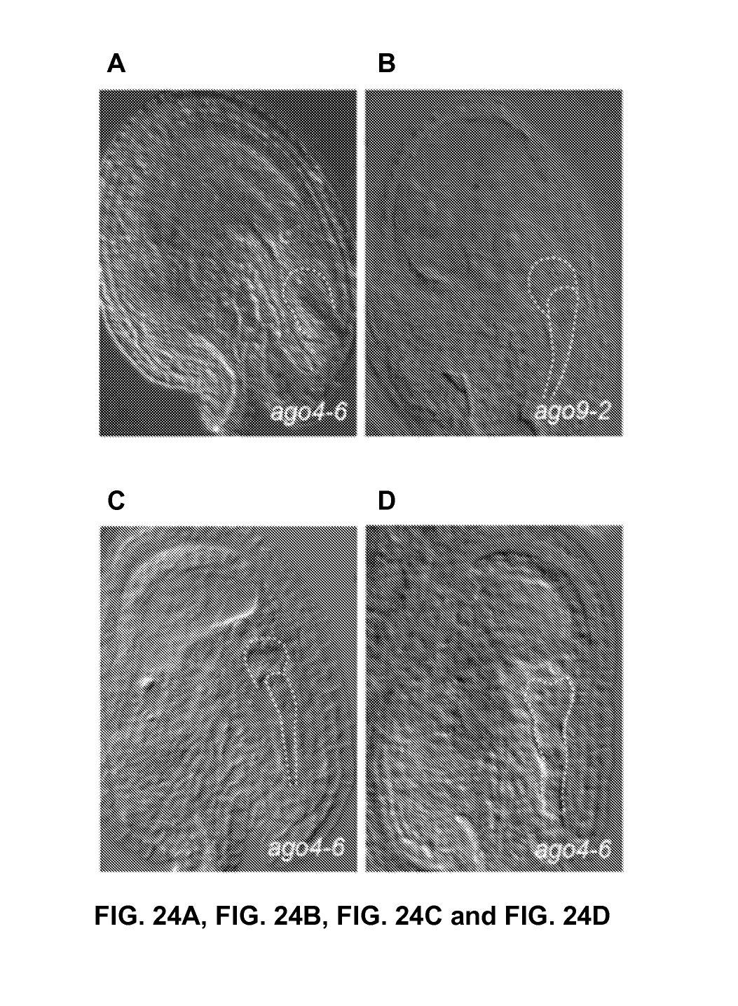

To determine if this autonomous formation of seeds is the result of a genetic interaction of pi with rdr6-15 and/or ago4-1, or if this phenotype is also present in single gene mutants involved in the RdDM pathway, immature flowers of rdr6-15/+, ago4-6/ago4-6 and ago9-2/ago9-2 individuals were emasculated, and resulting gynoecia were cytologically analyzed seven days after emasculation (dae). As in the case of plants showing the pi phenotype, the number of developing seeds that showed significant enlargement and prevalent turgidity compared to neighboring unfertilized ovules were quantified (FIGS. 7A to 7F; 16C; and 17A to 17C). In the case of twelve rdr6-15/+ individuals analyzed (116 emasculated flowers), the average percentage of seeds developing without pollination at seven dae was 26.39% (n=5849); in the case of six ago4-6/ago4-6 individuals (68 emasculated flowers), this same average was 37.93% (n=3200); and in the case of eight ago9-2/ago9-2 individuals (103 emasculated flowers), the average was 67.96% (n=4792; FIGS. 9-12). The cytological analysis of some of these seeds confirmed that in most cases they contain a developing embryo and endosperm with morphologies equivalent to wild-type (FIGS. 8 to 12; 3A to 31 and 18A to 18C). In a handful of cases, mature defective or normal seeds were recovered for all three mutants (FIG. 19A to 19D). Whole-mounted cleared specimens showed that non-fertilized ovules exhibit cytological evidence of seed coat initiation in the endothelial layer (FIG. 20A to 20C). Fertilization of a wild-type female gametophyte triggers the accumulation of proanthocyanidin pigments in the endothelium cell layer, which can be detected as a red stain by a vanillin assay (Debeaujon et al., 2003). In contrast to control unpollinated pistils, autonomously developing seeds showed red staining deposits in the endothelium, confirming that endosperm formation is activated (FIG. 21A to 21E). Many seeds developing in the absence of fertilization contained free nuclei in the central cell, reminiscent of normal endosperm development (FIG. 22A to 22C); for all three mutants the highest percentage was found at 7 dae (FIG. 23). Sporadically, developing seeds exhibited an early embryo (FIG. 24A to 24D). Finally, the analysis of non-pollinated siliques that reached full maturity revealed that all three genotypes are able to form viable seeds that germinate and give rise to diploid plants that are fertile. These results demonstrated that plants defective in RDR6, AGO4 or AGO9 can produce viable and fertile seeds in the absence of pollination.

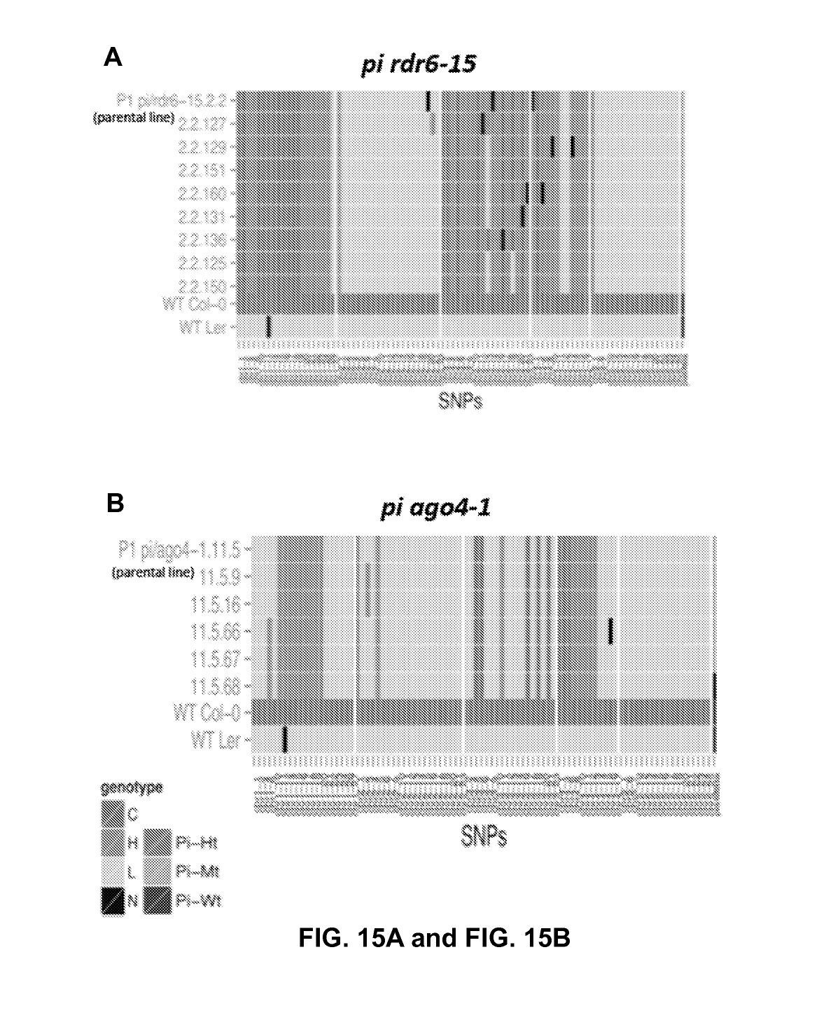

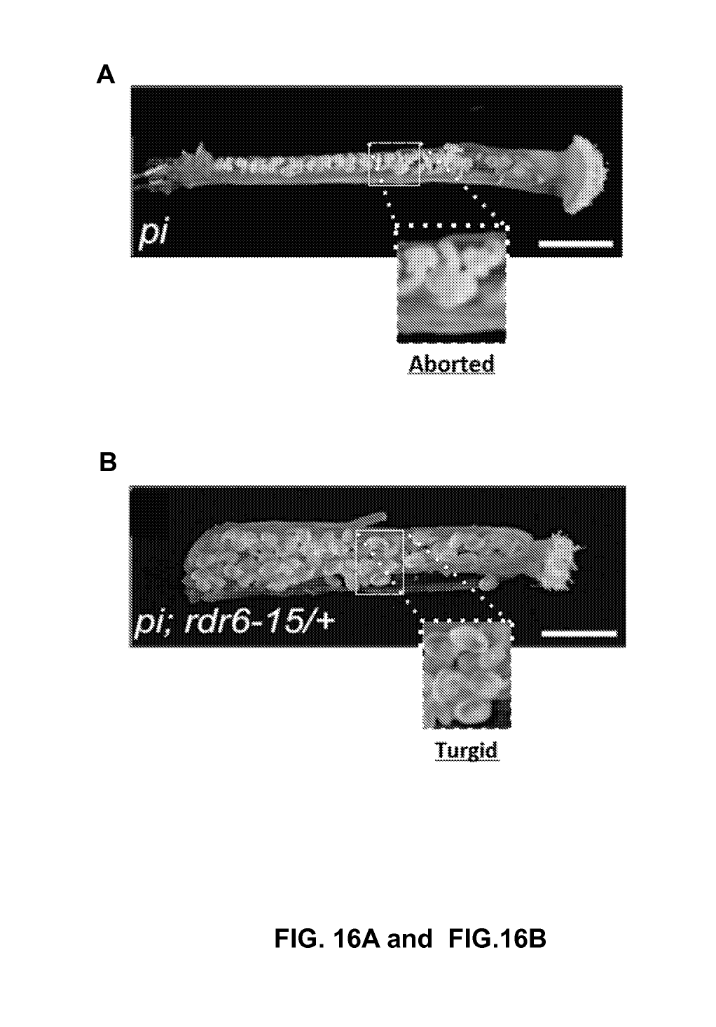

When germinated, seeds recovered from unpollinated pi rdr6-15/+, pi ago4-1/+, and pi ago4-1/ago4-1 individuals gave rise to diploid plants showing the pi phenotype. In the case of pi rdr6-15/+, the genetic background of the parental genotypes differed (pi is in a Landsberg erecta ecotype, whereas rdr6-15 is in Columbia-0), the chromosomal constitution of F3 individuals could be compared to the chromosomal constitution of their diploid progeny generated in the absence of pollination. In the case of ago4/+, a cross to Columbia-0 individuals allowed the establishment of ecotype heterozygosity to precede the subsequent generation of the pi ago4 individuals that were used for genotyping. To this aim, a collection of 89 molecular markers (SSLPs and CAPS) that recognize previously characterized allelic polymorphisms that distinguish both ecotypes across all five Arabidopsis chromosomes were used (Bell and Ecker, 1994; Cho et al., 1999; Lukowitz et al., 2000). Among 17 diploid F3 rdr6-15/+ individuals tested, 9 retained the heterozygosity and genotype of their mother at all tested loci and in all five chromosomes, and 8 were nearly clonal as they showed a single-locus polymorphism at different genomic locations (FIGS. 14 and 15). In the case of 13 diploid F3 ago4/+ individuals genotyped, 7 retained the heterozygosity and genotype of their mother at all tested loci and in all five chromosomes, and 5 were nearly clonal as they showed a single-locus polymorphism at different genomic locations (FIGS. 14 and 15). These results demonstrate that these two independent mutants can consistently produce clonal seeds.

The results show that self-propagated asexual reproduction through seeds can be induced in Arabidopsis by single mutations affecting genes involved in RNA-dependent DNA methylation, a discovery indicating that transcriptional repression of repetitive genomic regions, most often associated with heterochromatin, is essential for avoiding both ectopic gamete formation and the autonomous parthenogenetic activation of gametic cells (the egg and central cell). The mechanism uncovered is highly reminiscent of autonomous apomixis of the aposporous type, a phenomenon naturally occurring in flowering species of the genus Lamprothyrsus, and Hieracium (Bicknell et al., 2000). The frequency of autonomous initiation of seed development is superior to the frequency at which viable asexual seeds are recovered in the absence of pollination for both rdr6-15 and ago4-1. Non-pollinated siliques often contain seeds showing a multicellular embryo in the absence of free nuclear proliferation of the endosperm that eventually collapse, indicating that fertilization of the central cell might be necessary for the formation of viable asexual seeds by a mechanism reminiscent of pseudogamy in natural apomicts (Nogler, 1984). The results indicate the induction of self-propagated asexual reproduction through seeds by altering the activity of a single gene in cultivated crops.

2. Methods

i. Plant Material

Arabidopsis thaliana seeds of rdr6-15 (SAIL_34_G10), ago4-1 (CS6364), ago4-6 (SALK_071772), agog-2 (SALK_11205), and agog-3 (SAIL_34_G10) were germinated in Murashige and Skoog (MS) medium supplied with either kanamycin (50 ug/ul) or ppt (10 u/ul) and germinated in a growth chamber under short day conditions (16 hr light/8 hr dark) at 25° C. Seedlings were then transplanted to soil and grown in a greenhouse at 21° C.

ii. Genetic Analysis and Flow Cytometry

Adult double and single mutant individuals (pi rdr6, rdr6, pi ago4, ago4, and agog) were classified according to developmental stage, and siliques were numbered according to their sequential position in primary stems. The first flower that had fully lost additional floral organs was tagged as Silique#1, and subsequent older siliques were given a consecutive number. Individual siliques were dissected under a Leica stereomicroscope and scored for ovule abortion and developing seed formation. In the case of individuals showing a pistillata phenotype, all fully mature siliques were individually and systematically analyzed at maturity but before dehiscence with the help of magnifying glasses, keeping a register of the position of siliques containing at least one seed, and the number of seeds per silique. In the case of emasculated single mutant individuals, 3 to 4 immature flowers were emasculated before maturity, eliminating all stamens and floral organs except the gynoecium. Seven days after emasculation, each silique was dissected under a stereo-microscope and scored for aborted ovules and developings seeds.

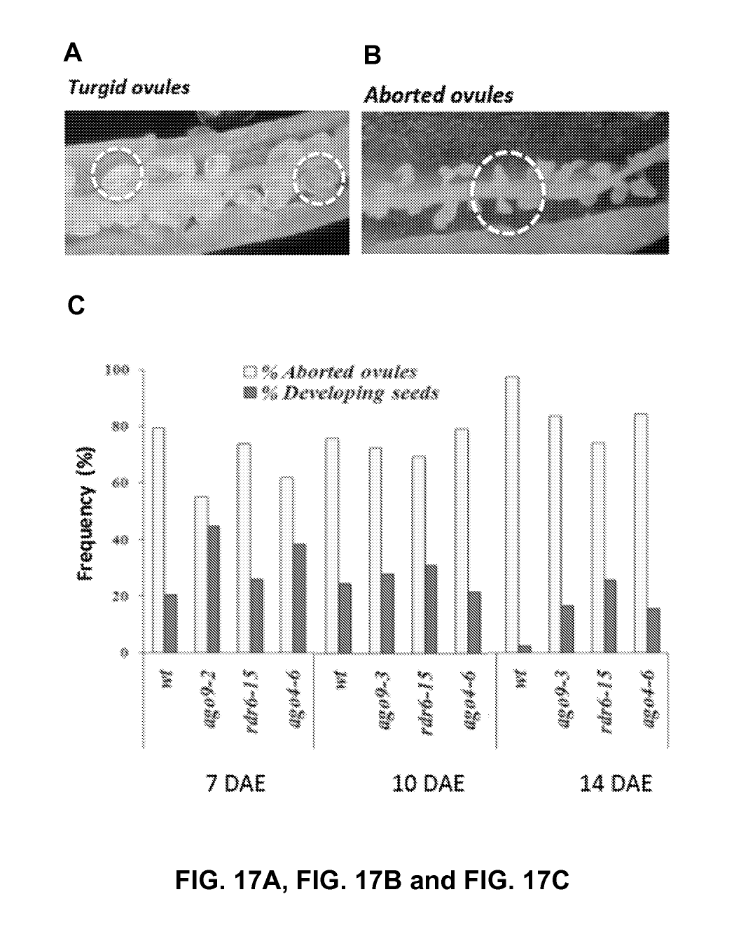

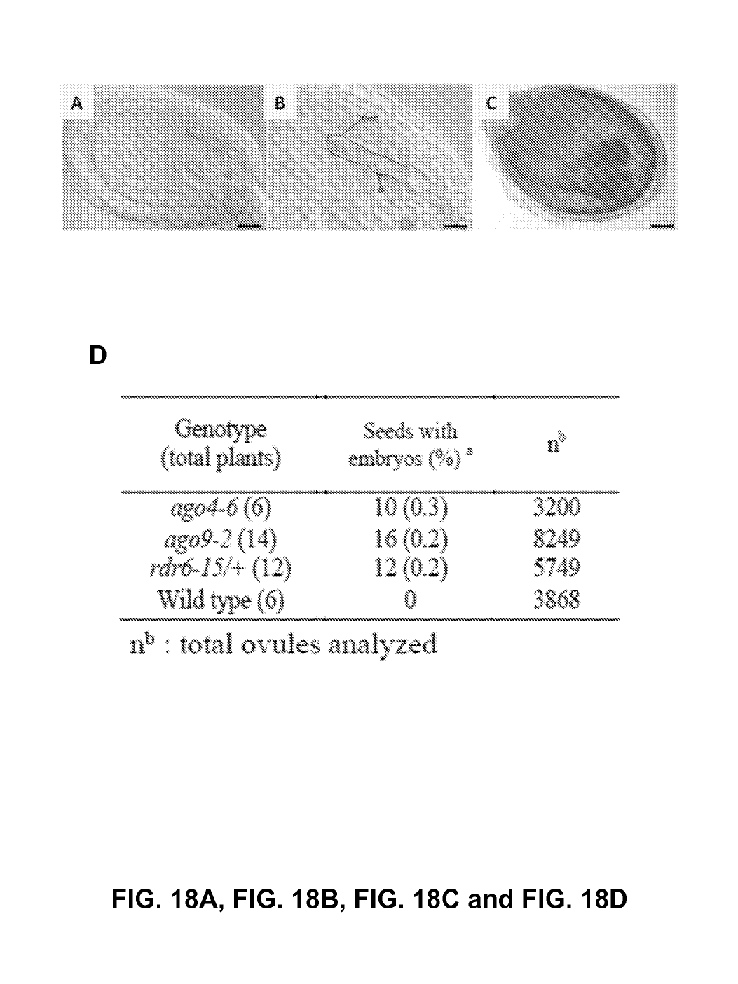

For flow cytometry, developing cauline leaves were used for all plants and processed as described in Dolezel et al 2007. DNA content was estimated with a PARTEC CUBE flow cytometer. Results of these methods are provided in FIGS. 2D, 5, 8, 9, 10, 11, 12, 13, 17C, 18D, and 19D.

iii. Whole-Mount Preparations and Histological Analysis

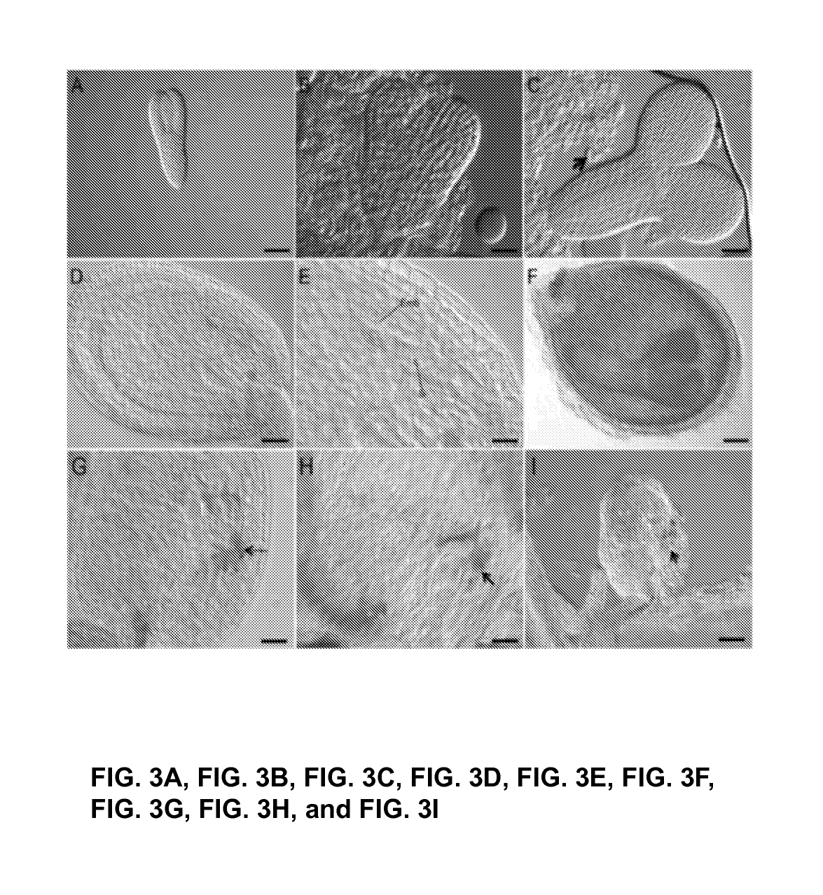

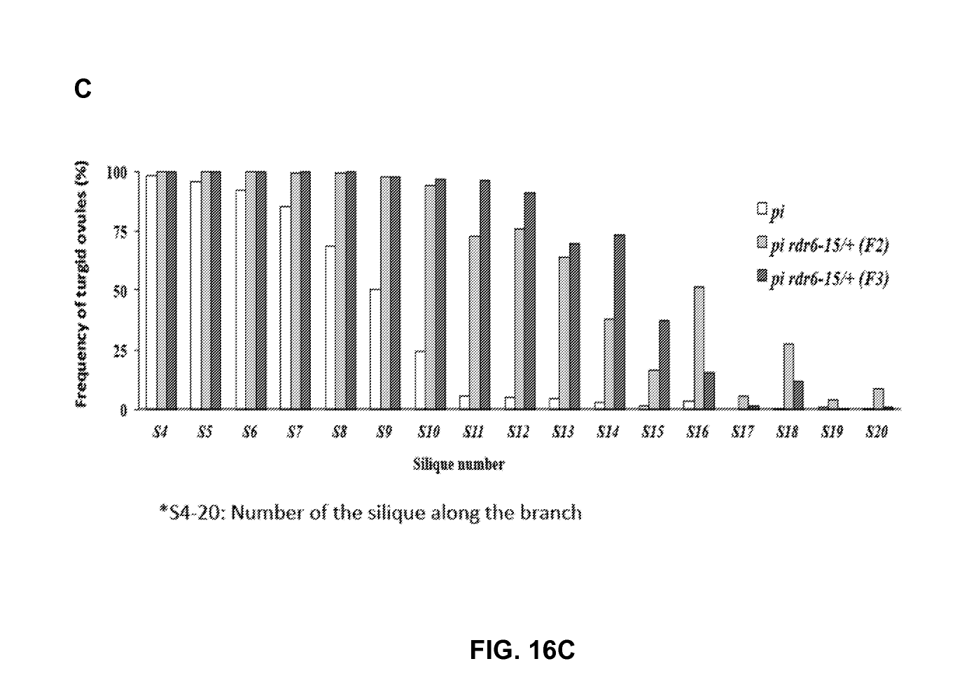

Developing siliques from wild-type, mutant or transformant lines were dissected longitudinally with hypodermic needles (1-mL insulin syringes; Becton Dickinson) and either fixed with FAA buffer (50% ethanol, 5% acetic acid, and 10% formaldehyde), dehydrated in increasing ethanol concentration, and cleared in Herr's solution (phenol:chloral hydrate:85% lactic acid:xylene:oil of clove [1:1:1:0.5:1]); or directly cleared in a drop of Hoyer's solution as previously described (Vielle-Calzada et al., 2000). For advanced stages of seed formation, seeds were punctuated to release the embryo; cleared seeds were observed in a Leica microscope (Wetzlar, Germany) under Nomarski optics. For vanillin staining, ovules and seeds were dissected from pistils and siliques, respectively, and incubated in an acidic solution (6 N HCl) of 1% (w/v) vanillin (Sigma-Aldrich) at room temperature (Aastrup et al., 1984), and mounted in a drop of glycerol. Vanillin reacts in the presence of proanthocyanidins that specifically accumulate in the endothelium layer of the seed coat, generating a red product (Debeaujon et al., 2003). Results of these methods are provided in FIGS. 2D to 2G, 3A to 31, 6A and 6B, 18A to 18C, 20A to 20C, 21A to 21E, 22A to 22C, and 24A to 24D.

iv. Molecular Genetic Analysis

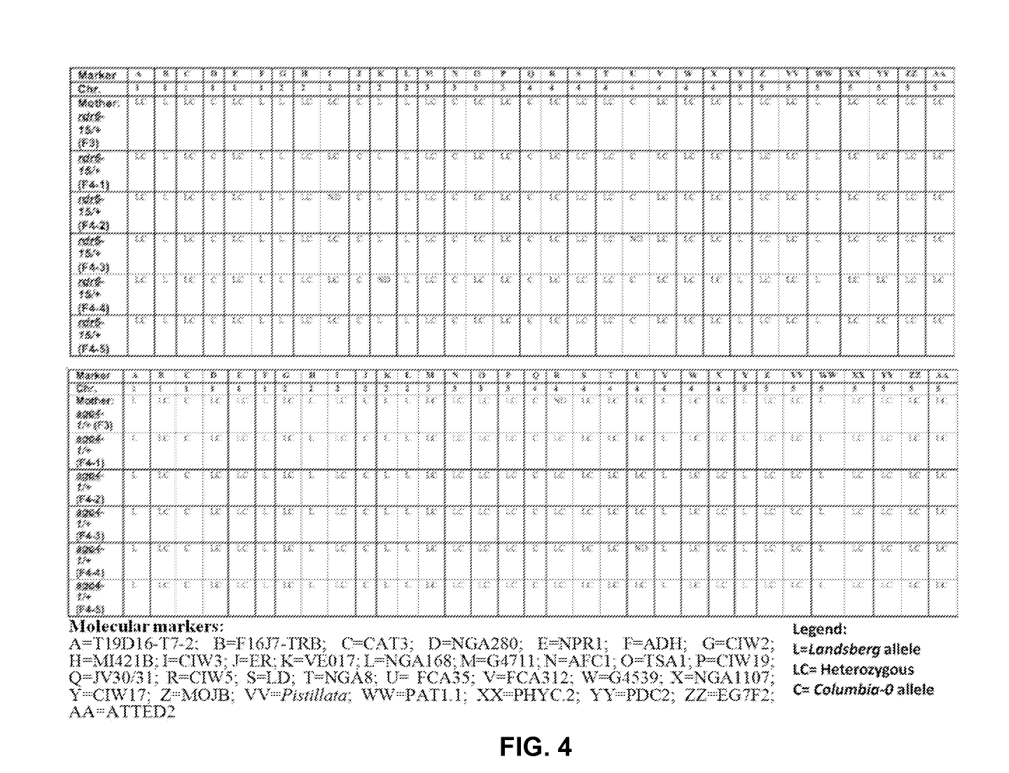

DNA extractions were performed as decribed in Vielle-Calzada et al., 1999; 1 μl of DNA was used for PCR amplification with 2 mM MgCl2, 0.2 mM of each dNTP, 1 U of Taq DNA polymerase (Invitrogen), 13 μL PCR buffer, and 20 pmol of each primer for 30 cycles at a variable annealing temperature depending on the marker as described for microsatellite markers and primer sequences obtained from TAIR (arabidopsis.org). Results of these methods are included in FIGS. 4, 14, and 15.

v. Genotyping

Seeds from all F3 individuals for both pi rdr6/+ and pi ago4/+ genotypes were germinated under growth chamber under short day conditions (16 hr light/8 hr dark) at 25° C. and total DNA extractions were conducted at rosette stage. Single nucleotide polymorphism (SNP) was obtained using a uniplex SNP genotyping platform for Kompetitive Allele Specific PCR (KASP), a global benchmark technology commercially provided by LGC Genomics (Beverly Mass. USA). Results of this method are provided in FIGS. 14 and 15.

### J. EXAMPLE 2

### Exemplary Embryo Markers and Exemplary Gametocides

1. Corn-Maize: An Embryo Marker for Detecting Monoploids of Maize (Zea Mays L.) D. K. Nanda and S. S. Chase

The use of an embryo marker of commercial value for detecting monoploids of maize is described. This system utilizes a male parent called the Purple Embryo Marker (b pl A C Rnj:Cudu or Pwf) which produces a deep purple pigment in the embryo and red or purple aleurone color in the endosperm. Kernels of a marked progeny which do nol exhibit purple color in the embryo but do have red or purple aleurone pigment are saved for the putative monoploid embryos contained. In this study, nine single crosses of commercial value were pollinated with the Purple Embryo Marker stock. Of the 194, 157 kernels classified, it was possible to discard more than 98% before germination, from 1-rom the kernels with putative monoploids 201 actual monoploids were realized. Purple Embryo Marker: produces a deep purple pigment in the embryo. Nanda D K and Chase S S. 1966. An embryo marker for detecting monoploids of maize. Crop Science Vol. 6 (2): 213-215.

2. Rice: Rod—Marks the Epidermal Layer of the Embryo. Kamiya N Nishimura A Sentoku N Takabe Nagato H Matsuoka M. 203. Rice Globular Embryo4 (Gle4) Mutant is Defective in Radial Pattern Formation During Embryogenesis. Plant Cell Physiology 44(9): 875-83

REES and REE8—only expressed in the whole embryo

Reference: Kikuchi K, Chung C S Yoshida K. 1998. Isolation and Analysis of New Molecular Embryogenesis in Rice (Oryza sativa L.). Plant Biotechnology 15(2):77-81.

goliath—embryo size marker, the mutation makes the embryos larger

Reference:

Kawakatsu T Taramino G Itoh J allen J Sato Y Hong S K Yule R Nagasawa N goliath—embryo size marker, the mutation makes the embryos larger

Reference: Kawakatsu T Taramino G Itoh J allen J Sato Y Hong S K Yule R Nagasawa N Kojima M Kusaba M Sakakibara H Sakai H Nagato Y. 2009.

PLASTROCHRON3/GOLIATH encodes a glutamate carboxypeptidase required for proper development in rice. Plant Journal 58(6):1028-1040

3. Wheat:

Anthocyanin Purple Pigment

Reference: K. M. Doshi, F. Eudes, A. Laroche, and D. Gaudet. 2007. Anthocyanin expression in marker free transgenic wheat and triticale embryos. Vitro Cellular and Developmental Biology—Plant, 43(5):429-435

4. Sorghum:

Promoter pPZP201 fused to GFP as a reporter gene

Reference: Gurel S Gurel E Miller t Lemaux P G. 2012. Agrobacterium-Mediated Transformation of Sorghum bicolor Using Immature Embryos. In Transgenic Plants; Methdos and Protocols Series No. 847: 109-122

5. Sunflower:

p35CaMV::uida fusion

Reference: Burrus M Molinier J Himber C Hunold R Bronner R Rousselin P Hahne G. 1996. Agrobacterium-mediated transformation of sunflower (helianthus annuus L.) shoot apices: transformation patterns. Molecular Breeding 2; 329-338.

6. Coffee:

p35CaMV::GFP fusion

Reference: Mishra M Devi S McCormac Scott N Chen D Elliott M Slater A. 2010.

Green fluorescent protein as a visual selection marker for coffee transformation. Biologia 65:639-646.

7. Faba Bean:

Natural mutation il-1—Green cotyledons.

Reference: G. Duct, F. Moussyl, X. Zong2, G. Ding. 1999. Single gene mutation for green cotyledons as a marker for the embryonic genotype in faba bean, Vicia faba. Plant Breeding 118 (6):577-578.

8. Arabidopsis:

ET1275—Encodes a COP1-interacting protein named CIP8; ET1278—Encodes a isoflavone reductase homolog. (Vielle-Calzada Jp, Baskar R, and Grossniklaus U. 2000. Delayed activation of the paternal genome during seed development. Nature 404: 91-94.)

9. Plant Gametocides

maleic hydrazide (MH) (WITTWER, S. H., and HILLYER, I. G., 1954. Chemical induction of male sterility in Cucurbits. Science, 120, 893-894.); 2,4-dichloropheno-xyacetic acid (2,4-D) (REHM, S., 1952. Male sterile plants by chemical treatment. Nature 170, 38-39.); a-naphthalene acetic acid (NAA) (LAIBACH, F., and KRIBBBN, F. J., 1950. Der Einfluss von Wuchsstoff auf die Blutenbildung der Gurke. Naturwuienschaften 37, 114.); ri-iodoben2oic acid (TIBA) (ATON, F. M., 1957. Selective gametocide opens way to hybrid cotton. Science, 1174-1175)

### ADDITIONAL REFERENCES ON GAMETOCIDES

Albertson, M. C. and Palmer, R. G., 1979, A comparative light and electron microscopic study on macrosporogenesis in male sterile (MS) and male fertile soybeans (Glycine max L. Var.). American J. Bot., 66: 253-265.
Ali, A. J., Devakumar, C., Zaman, F. U. and Siddiq, E. A., 1999, Gametocidal potency of ethyl 4 fluorooxanilate in rice. Indian J. Genet. Pl. Bred., 59: 267-279.
Alpuerto, V. V., 1987, Inducted male sterility using gametocides in the production of hybrid tomato seed. Philippine J. of Crop Sci., 12: 20.
Anaschenko, A. V., 1972b, A study of male sterility in sunflower. Tr. Prikl Bot. Genet Sel., 46: 120-13.
Anonymous, 1999, International Rules for Seed Testing, Seed Science and Technology, 32: 1-334.
Anonymous, 2007, Agricultural output. Centre for Monitoring. Indian Economy, pp. 267-268.
Ashwathanarayana, S. C., 1986, Effect of gametocides on pollen sterility and other morphological characters in rice (Oryza sativa L.). M. Sc. (Agri.) Thesis, Univ. Agric. Sci., Bangalore.
Awasthi, N. N. C. and Dubey, D. K., 1985, Effects of some phytogametocides on growth, fertility and yield of Lentil (Lens culinaris). LENS News. (ICARDA), 12: 16-22.
Banga, S. S. and Labana, K. S., 1984, Ethrel induced male sterility in Indian mustard (brassica juncea (L.) Coss). Zpflanzenzucht, 92: 229-233.
Bartkawiak, B. I., Rousselle, P. and Renard, M., 1979, Investigations of two lines of cytoplasmic male sterility in rape seed (Brassica napus L.). Genetica polanica, 20: 47-49.
Basavaraja, N., 1981, Studies on hybrid seed production in brinjal (Solanum melongena L.). M. Sc. (Agri.) Thesis, Univ. Agric. Sci., Bangalore.
Baydar, H. and Gokman, O. H., 2003, Hybrid seed production in safflower following the induction of male sterility by gibbberellic acid. Pl. Bred., 122: 459-461.
Beaumont, V. and Courtois, B., 1990, Anther culturability of rice plants treated with male gametocide chemicals. Internl. Rice Res. News., 15:9.
Bennekom, J. L. and Van, 1973, Application of gibberellic acid as a gametocide in onions. Zaadbelangen, 27(16): 324-325.
Berry, S. K., Kalra, C. L., Sehgal, R. C., Kulkarni, S. G., Arora, S. K. and Sharma, B. R., 1988, Quality characteristics of seed of five okra cultivars. J. Food Sci. Tec., 25: 303-305.
Berzy, T., Szundy, T., Barnabas, B., Bayer, K. and Matolesy, G., 1990, The biological effect of gametocidies on sexual processes and individual plant development in maize. Novenytermeles, 39: 97-110.
Bhardwaj, D. N. and Dubey, D. K., 1977, Chemical induction of male sterility in mungean (Phaseolus aureus Roxb.). Sci. Cul., 43(2): 89-92.
Bose, S. and Sharma, P. D., 1972 Preliminary studies on the effect of combined treatment of colchicine and giberelic acid on rice. Agric. Agro-Indus. J., 5(2): 31-32.
Campos, F. A., 1974, The use of ethrel in the induction of male sterility in sunflower. In Proceedings of the 6th Internl. Sunflower Conf., vraneanu A.V. (Ed.) Bucharest, pp. 349-351.
Chailakeryan, M. K. H. and Khryanin, V. N., 1980, Hormonal regulation of sox expression in culture of isolated hemp germs. Naturwissens Chaften, 67: 94-96.
Chan, Y. W. and Cheah, C. H., 1981, Evaluation of ethrel as a selective gametocide in the breeding of rice (Oriza sativa L.). In Proceedings of 4th International SABRAO Cong., Malaysia, p. 24.
Chauhan, S. V. S. and Kinoshita, T., 1980a, Morphological and histochemical studies on pollen degeneration in cytoplasmic male sterile sugarbeet (Beta vulgaris L. Var Saccharifera). J. of Fac. Agric. Hokkaido Univ. Japan, 60: 42-46.
Chauhan, S. V. S. and Kinoshita, T., 1980c, Cytohistological and biochemical studies on pollen abortion in Datura alba L. plants treated with gametocidal compounds. In Proceedings of Japanese Acad. of Sci., 56: 344-349.
Chauhan, S. V. S. and Singh, S. P., 1966, Pollen abortion in male sterile hexaploid wheat (Norin) having Aegilaps ovota L. cytoplasm. Crop Sci., 6: 532-535.
Chauhan, S. V. S. and Singh, S. P., 1972, Effect of maleic hydrazide, FW 450 and dalapon on growth, flowering and pollen viability of Capsicum annuum L. and Datura alba L. Indian J. Pl. Phy., 15: 138-147.
Chauhan, S. V. S. and Vandana Singh, 2002, Detergent induced male sterility and bud pollination in Brassica juncea (L.). Czem and Coss. Curr. Sci., 82(8): 918-920.
Chauhan, S. V. S., 1976, Morphological and histochemical studies in natural and chemically induced male sterile plants. Curr. Sci., 45; 274-275.
Chauhan, S. V. S., 1980, Effect of maleic hydrazide, FW-450 and dalapon on anther development in Capsicum annuum. J. Indian Bot. Sci., 59: 133-136.
Chauhan, S. V. S., Agnihotri, D. K. and Gupta, H. K., 2005, Efficacy of benzotrizole as a chemical hybridizing agent in chilli cotton radish. Indian J. Genet., 65(3): 223-224.
Cheng, Y. K. and Huang, C. S., 1980, Studies on cytoplasmic genetic male sterility of cultivated rice (Oryza sativa). II. Morphological histological investigation of functional male sterility. J. of Agri. Res., China, 29(29): 7-8.
Chopra, V. L., Jain, S. K. and Swaminathan, M. S., 1960, Studies on the chemical induction of pollen sterility in some crop plants. Indian J. Genet. Pl. Breed., 20: 188-199.
Choudhury, B. and George, P. V., 1962, Preliminary trails on the induction of male sterility in brinjal (Solanum melongena L.). Indian J. Hort., Sci., 19: 140-142.
Chowdhury, J. B. and Das, K., 1966, Male sterility in Brassica campestris var. yellow sarson. Indian J. Genet and Pl. Bred., 26: 374-380.
Chowdhury, J. B. and Das, K., 1968, Cytomorphological studies on male sterility in Brassica campestris L. Cytologia, 38: 195-199.
Chowdhury, J. B., Ghai, B. S. and Varghese, T. M., 1968, Cytohistological studies on male sterility in wheat with a discussion on causes of pollen fertility in other crops. Indian J. Genet Pl. Bred., 20: 188-199.
Ciha, A. J. and Ruminski P. G., 1991, Specificity of pyridine monocarboxylates and benzoic acid analogues as chemical hybridizing agents in wheat. J. Agri. Food Chem., 39: 2072-2076.
Colhoun, L. W., Steer, M. V., 1983, The cytological effects of the gametocides ethrel and RH 531 on microsprogenesis in barley (Hardeum vulgare L.). Pl. Cen Environ., 196(1): 21-29.
Das, K. and Pandey, B. D., 1961, Male sterility in brown sarson. Indian J. Genet Pl. Bred., 21: 195-190.
Deotale, R. D., Bhiwapukar, R. M., Sorte, N. V., Waghmare, H. U., Nimje, B. H. and Alluwar, M. W., 1994, Growth and yield components of safflower (Carthamum tinctiorius L.) as influenced by 2, 3,5-triiodobenzoic acid. J. Soils and Crops, 4: 125-127.
Dharmarayan, D. K., 1990, Chemical induction of male sterility and standardizing techniques for hybrid seed production rice (Oryza sativa L.). Ph.D. Thesis, Univ. Agric. Sci., Bangalore.
Dicks, J. W., 1976, Chemical restriction of stem growth in ornamentals. Outlook on Agri., 9: 69-75.
Dubey, R. S. and Singh, S. P., 1967, Chemical induction of male sterility in Abelmoschus esculentus (L.) Moench. Indian J. Agric. Sci., 38(1): 108-114.
Dutta, O. P., 1980, Male sterility in okra (Abelmoschus esculentus L. Moench) and bottle gourd and its utilization in hybrid seed production. Ph.D. Thesis, Univ. Agri. Sci., Bangalore.
Echlin, P., 1971, The role of tapetum during microsporogenesis of angiosperms, in Pollen Development and Physiology. Heslop-Harrison (ed.), Butterowrths, London, pp. 41-61.
Eenink, A. H. and Vereijken, A. L. J., 1978, Anatomical changes in flower buds of lettuce (Lactuca sativa L.) treated with a GA3 solution for induction of male sterility. Acta Bot Neerl., 27: 199-224.
Erickson, J. R., 1967, Biochemical investigations of cytoplasmic male sterility in spring wheat (Triticum aestiuvm L.). Agron. Abst., pp. 8-9.
Fairey, D. T., Shoskopf, N. C., 1975, Effect of granevar ethephon on male sterility in wheat. Crop Sci., 15(1): 29-32.
Fan Ping, Cui Dangqun and Fan Hong Wei, 1998, Studies on the male sterility induced by CHA-SC 2053 in common wheat. Acta Agriculturae Universitatis Henanensis, 32(2): 149-153.
Frank, J. and Koves, F. S., 1977, Chemical induction of male sterility in sunflower. Acta Agrono. Acad. Scienti. Hungarical, 26(3/4): 318-324.
Frank, J., Koves, F. S., 1977, Chemical induction of male sterility in sunflower. Acta Agron. Acad., Scien. Hung., 26(/4): 318-324.
Gao, Q. R., Sun, L. and Liu, B., 1996, Induced male sterility and its effects on growth and development of winter wheat. J. of Shandong Agri. Univ., 27: 241-248.
Garcia, T. L., Dominguez, J. and Fernandez Martinez, J., 1979, Male sterility and female steility induced in sunflower with GA3. Anales del Institute Nacional de investigeones Agraries Prod Vegel., 9: 147-169.
Ghosh, M. S. and Bose, T. K., 1970, Sex modification in cucurbitaceous plants by using CCC. Phyton. (Hom), 27: 131-135.
Gomez, A. K and Gomez, A., 1984, Statistical Procedure for Agricultural Research, 2nd Edition, A Wiley—Interscience Publication, New York, pp. 187-241.
Graybosch, R. A. and Palmer, R. G., 1987, Analysis of male sterile character in soybean. J. Here., 78: 66-70.
Guan, C. Y., Li, X., Wang, G. H. and Chen, S. Y., 1998, Studies on the mechanisms of male sterility induced by chemical hybridizing agents in rape. II. Effects of KMS-1 on fertility in rape (Bassica napus). Chinese Journal of Oil and Crop Science, 20: 1-5.
Guilford, W. J., Patterson, T. G., Vega, R. O., Fang, L., Liang, Y., Lewis, H. A. and Labovitz, J. N., 1992, Synthesis and pollen suppressant activity of phenylcinoline-3-carboxylic acids. J. Agri. Food Chem., 40: 2026-2032.
Halevy, A. H. and Rudich, J., 1967, Modification of sex expression in muskmelon by treatment with the growth retardant B 995. Physiologia Plantarum, 20: 1052-1058.
Hansen, D. J., Bellman, S. K. and Sacher, R. M., 1976, Gibberellic acid controlled sex expression of corn tassels. Crop Sci., 16: 371-374.
Hecker, R. J. and Smith, G. A. 1975, Tests of granular ethephon as a male gametocide on sugarbeet. Canadian J. Pl. Sci., 55: 655-656.
Hecker, R. J., Bilgen, T., Bhatnagar, P. S., Smith, G. A., 1972, Tests for chemical induction of male sterility in sugarbeet. Canadian J. Pl. Sci., 52(8): 937-944.
Hillyer, I. G., 1956, Effects of growth substances in flowering of cucurbitaceous plants. Ph.D. Thesis, Michigan State University.
Huang, Q. C. and Wang, L. Z., 1990, Use of male gametocide to induce complete sterility in a partially male sterile rice. Internl. Rice Res. News., 15(5): 6-7.
Huang, X. Q., Yang, A. N., Zhou, Y. L. and Zhang, Y. J., 1999, Effect of chemical hybridizing agent III (Pyrone type derivative) on male sterility in rice. Jiangsu J. Agri. Sci., 15(1): 17-20.
Hughes, W. G., Bodden, J. J. and Golanoponlu, S., 1978, The effect of sowing density and application of GA on male sterility and ear emergence in Ethephon treated field grown wheat. Ann. Rev. Ap. Bio., 88: 313-319.
Iang, M. L. L., Wang, D. Q., Zhang, A. and Huang, C., 1998, Male sterile effect of a new pyridazine compound 9403 on wheat. J. China Agri. Univ., 3: 39-44.
Illusulu, K., 1967, Cytological investigation on the male sterility in sunflower. Genetica Polanica, 30: 65-69.
Iwahori, S., Lyons, J. M. and Smith, O. E., 1970, Sex expression in cucumber plants as affected by 2-chloroethylphosphonic acid, ethylene and growth regulators. Plant Physiology (Bethesda), 46: 412-415.
Jackson, M. L., 1967, Soil Chemical Analysis. Prentice Hall of India Private Limited, New Delhi, pp. 183-192.
Jain, S. K., 1956, Natural incidence of male sterility and its chemical induction in crop plants. Assoc. Disst., IARI, New Delhi, p. 267.
Jain, V. K. and Mukherjee, D., 1980, Effect of chlorflurenol methylester 74050 on sex expression and parthenocarpic fruit development in tomato. Phyton. (Hom.), 38: 89-93.
Jensen, W. A., 1962, Bot. Histochem. Prin. Prac., W. H., Freeman and Co., San Francisco.
Jensen, W. A., 1984, The effect of chemical hybridizing agent on the development of wheat pollen. In Proceedings of 8th Internl. Sym. Sexual Reprod. In Seed Pl., Fems and Mosses, p. 34.
Jiang, M. L., Wang, D. Q., Zhang, A. and Huang, C., 1998, Male sterile effect of a new pyridazine compound 9403 on wheat. J. of China Agri. Univ., 3(5): 39-44.
Johnson, R. R. and Brown, C. M., 1976, Chemical control of pollination in wheat and oats. Crop Sci., 16: 584-587.
Jos, J. S. and Singh, S. P., 1967a, Chemical induction of male sterility in tobacco. Indian J. Agri. Sci., 37: 504-510.
Jos, J. S. and Singh, S. P., 1967b, Induction of male sterility in Nicotiana rustica by means of chemicals. Indian J. Pl Phy., 10: 130-138.
Joshi, A. D. and Hardas, M. W., 1974, Okra In: Hutchnson, J. B. Evolutionary Studies in World Crops. Diversity and Change in the Indian Sub-Continent. Cambridge Univ., Press, London, p. 105.
Kajjidoni, S. J., 1997, Histological basis of genetic male sterility and its utilization in hybrid development in diploid cotton. Ph.D. thesis, Univ. Agri. Sci., Dharwad.
Kasambe, J. N. R., 1967, Phenotypic restoration of fertility in a male sterile mutant by treatment with gibberellic acid. Nature, 215: 668.
Kaul, C. L. and Singh, S. P., 1967a, Effects of some growth regulators with gametocidal properties on Cajanus cajan L. Indian J. of Agri. Sci., 37: 69-75.
Kaul, C. L. and Singh, S. P., 1967b, On induced male sterility in wheat, Sunnhemp and onion. Indian J. of Pl. Phy., 10: 112-118.
Kaul, C. L. and Singh, S. P., 1967c, Staminal and functional male sterility induced by chemical treatment in papilionaceous plants. Indian J. Agri. Sci., 37: 264-269.
Khulbe, R. K., Roy, N. and Yadav, V. K., 2003, Induction of male sterility in wild and related species of sunflower (Helianthus annus L.). Crop Sci., 131: 29-32.
Kiermayer, O., 1959, Induktion mannlich-steriler Bluten bei Helianthus annuus durch, 2, 3, 5-tri-jodbenzoesaure (TIBA). Nature Wissen., 46: 457.
Kini, A. V., Seetharam, A. and Joshi, S. S. 1994, Mechanism of pollen abortion in cytoplasmi male sterile line of sunflower. Cytologic, 54: 121-124.
Kinoshita, T., 1971, Genetical studies on the male sterility of sugarbeets (Beta vulgaris L.) and its related species. J. of Fac. Agri., Hokkaido Univ., 56: 432-541.
Klimov, M. N., 1973, Induction of male sterility in sunflower by a chemical method. Referat Zhu. Abst., 5: 55-57.
Knowles, G., 1953, Selective control of wild oats in cereal crops by Maleic hydrazide. Canadian J. Agri. Sci., 33: 402.
Konstantinova, L. N., 1980, Cytological disturbances in anther development following gametocide induced male sterility in sunflower (Helianthus annus L.). Genetika a Lechenl, 67(3): 134-140.
Konstantinova, L. N., 1980, Cytological disturbances on anther development following gametocide induced male sterility in sunflower (Helianthus annus L.). Genetika a Slechenl, 67(3): 134-140.
Koriesh, E. M., Abou, Dahab, A. M. and Ali, E. W. M., 1989, Physiological studies on Chrysanthemum morifolium. Effect of cycocel, GA and nucleic acid on flowering and inflorescence characters. Assiut J. Agric. Sci., 20(1): 43-58.
Kovacik, A. and Kryzanek, R., 1969, Biological and biochemical analysis of sterile and fertile pollen of sunflower. Genet. Slecht., USSR, 5: 79-184.
Kumar, J., Verma, M. M., Singh, K. and Gagneja, M. R., 1976, Effectiveness of ehrel as an androcide in barley. Crop Improv., 3: 39-42.
Laibach, F. and Kribben, F. J., 1951, Der Einfluss von wuchstoff auf das Geschiecht der Bluten be einer monoezischen pflanze (Cucumis sativus L.). beitr Biol. Pflanz., 28: 64-67.
Lal, S. K., Devkumar, C., Sapra, R. L., Singh, K. P., 2004, Use ofgametocide for emasculation in soybean (Glycine max (L.) Men.). Soybean Gene. Newsl.: 31: 1-4.
Laveau, J. H., Schneider, C. and Berville, A., 1989, Microsporogenesis and abortion in cytoplasmic male sterile plants from H. petiolans or H. petiolaris fallan crossed by sunflower (H. annuus L.). Ann. Bot., 64: 137-148.
Lippert, L. F. and Hall, M. C., 1961, Gametocidal action of F W-450 on cantaloupe. In Proceedings of American Soc., Hort., Sci., 78: 319-329.
Liu, Q. L. Pen, L. S., Lu, X. Y., Luo, Z. M., Lin, F. S., Zhoo, T. B. and Wu, S. Q., 1998, Studies on utilizing a chemical hybridizing agent to guarantee the purity of two line hybrid rice. II. Baochuling's effect on TPGMSR's physio-chemical characters. J. Hunan Agri. Univ., 24: 345-350.
Mangal, J. L., 1972, Induced male sterility in brinjal following foliar application. Punjab Hort J., 12: 179-182.
Manjula, M. and Ibrahim, K. K., 1999, The effect of gametocides on plant growth characters in rice. Oryza, 36(3): 215-218.
Martin, F. W., 1982, A second edible okra species and its hybrid with common okra. Ann. Bot., 50: 277-283.
Mathur, D. S. and Lal, S. K., 1999, Chemical induction of male sterility in chickpea. Indian J. Genet Pl. Breed., 59(3): 379-380.
Mcllrath, W. J., 1957, The use of maleic hydrazide for the production of male sterility in grain sorghum. In: A. Literature Summary on Maleic hydrazide, Naugatuck Chemical Division, US Ruber Conn., p. 106.
McRae, D. H., 1983, Advances in chemical hybridization. Pl. Bred. Rev., 3: 169-191.
Meer, O. P. and Bennekam, J. L., 1973, Gibberellic acid as a gametocide for the common onion (Allium cepa L.). Euphy., 22(2): 239-243.
Mehetre, S. S., 1988, Male sterility in cotton. Agril. Rev., 9(1): 7-17.
Meyer, J R., Roux, J. B. and Thomas, R. O., 1958, A preliminary report on the induction of male sterility in cotton by maleic hybrzaide. Missouri Agril. Exper. Sta. Inf. Sheet, p. 589.
Mian, H. R., Kuspira, J. and Walker, G. W. R., 1974, Histological and cytochemical studies on five genetic male sterile lines of barley (Hardeum vulgare). Canadian J. Genet Cyt., 16: 355-379.
Miller, D. A., 1961, A study of various chemicals as gametocidal agents in sorghum. Sorghu News., 4: 22-24.
Mohan Ram, H. Y. and Jaiswal, V. S., 1970, Induction of female flowers on male plants of Cannabolis sativa L. by 2 chloroethanephosphonic acid. Experimentia (Basel), 26: 214-216.
Moore, J. F., 1959, Male sterility induced in tomato by sodium 2,3-dichloroisobutyrate. Science, 129: 1738-1740.
Moore, R. H., 1950, Several effects of maleic hydrazide on crop plants. Crop Sci., 112: 52-53.
Nachalas, M. M., Crawford, D. T., Goldstein, T. P. and Seligman, A. M., 1958 The histochemical demonstration of cytochrome oxidase with new reagent for the Nadi reaction. J. Histochem.
Cytochem., 6: 445-456.
Nagarjuna, B., Reddy, V. P., Rao, M. R. and Reddy, E. N., 1988, Effect of growth regulators and potassium nitrate on growth, flowering and yield of chrysanthemum (Chrysanthemum indicum). South Indian Hort., 36(3): 136-140.
Nakashima and Hosokawa, S., 1974b, Studies on histological features of male sterility in sunflower (Helianthus annus L.). Proc. Crop Sci. Soc. Japan, 43: 475-481.
Narayana Gowda, J. V., 1990, Influence of pinching and cycocel on growth and flowering of china aster (Callistephus chinensis). Mysore J. Agric. Sci., 24: 278-245.
Nasrallah, M. E. and Hopp, R. J., 1963, Effect of a selective gametocide on egg plant (Solanum melongena L.). In Proceed. of American Soc. Horti. Sci., 83: 575-578.
Nelson, P. M. and Rossman, E. C., 1958, Chemical induction of male sterility in inbred maize by use of gibberellins. Science, 127: 1560-1501.
Nishi, S., Kuriyan and Tode, M., 1970, Studies on first generation hybrids and vegetables II. Experiments on the applications of gametocides, FW 450 to tomato. Bull. Hort. Res. Stat., Hirat., 9: 129-139.
Olvey, J. M., Fisher, W. D. and Patterson, L. L., 1981, TD 1123: a selective male gametocide. In Proc. Beltwide Cot. Prod. Res. Conf., Ed. J. M. Brown, Memphis, Tenn.: National Cotton Council of America, p. 84.
Panse, V. G. and Sukhatme, P. V., 1978, Statistical Methods for Agricultural Workers, ICAR, New Delhi.
Parmar, K. S. Siddiq E. A. and Swaminathan, M. S., 1979 Chemical induction of male sterility in rice. Indian J. Genet Pl. Breed., 39: 529-541.
Paun, L., 1974, The cytologic mechanism of male sterility in sunflower. In Proc. 6th Intern. Sunf. Conf., Buchares, pp. 17-25.
Pederson, M. W., 1959, Effect of sodium 2,3-dichloroisobutyrate on alfalfa gametes. Agron. J, 51: 573-574.
Peiretti, D. A., Ceballos, H., Macchiavelli, R. E. and Fernandez, M., 1987, Effects of inducing male sterility by applying GA3 and F1 seed production in sunflower. Revista de Ciencias Agropecuarias, 5: 25-33.
Perez, A. T., Chang, T. T., Beachell, H. M., Vergara, B. S. and Marciano, A. P., 1973, Induction of male sterility in rice with ethrel and RH-531. SABRAO Newsletter, 5: 133-139.
Piquemal, G., 1970, How to produce hybrid sunflower seeds by inducing male sterility with gibberellic acid. In Proc. 4th Internl. Sunf. Conf, Memphis tennesses, pp. 127-135.
Porter, K. B. and Weise, A. F., 1961, Evaluation of certain chemicals as selective gametocides for wheat. Crop Sci., 1: 381-182.
Prayaga, P., Lakshamma and Anjani, K., 2002, Enhancement of male sterility in safflower by growth regulators and chemicals. Sesame and Safflower Newsletter, 16: 92-95.
Pundir, N. S. and Singh, S. P., 1965, Induction of male sterility in muskmelon by the use of FW-450. Agra Univ. J. Res. Sci., 14: 177-184.
Radley, M., 1980, Effect of abscicic acid and gibberellic acid on grain set in wheat. Ann. Rev. App. Bio., 95(3): 409-414.
Raj, A. Y., 1968, Histological studies in male sterile and male fertile sorghum. Indian J. Genet. Pl. Bred., 28: 335-341.
Rastogi, R. and Sawhney, V. K., 1988, Suppression of stamen development of CCC and ABA in tomato floral buds cultured in vitro. J. P1 Phy., 133: 620-624.
Rehm, S., 1952, Male sterile plants by chemical treatments. Nature, 170: 38-39.
Reinecke, D. M. and Bandurski, 1987, Auxin biosynthesis and metabolism. Plant Hormones and their role of plant growth and development. Ed. Devies, P. J., Boston, Martinus, Nijhoff, pp. 24-42.
Robinson, R. W., Whitaker, T. W. and Bohn, G. W., 1970, Promotion of pistillate flowering in cucurbiat by 2-chloro-ethyl phosphonic acid. Euphytica, 19: 180-183.
Rodriquez, B. P. and Lambeth, V. N., 1972, Synergism and antagonism of GA and growth inhibitors on growth and sex expression in cucumber. J. American Soc. Hort. Sci., 97: 90-92.
Rudich, J., Halevy, A. H. and Kedar, N., 1969, Increase in femaleness of three cucurbits by treatment with ethrel, an ethylene releasing compounds. Planta (Berl.), 86: 69-76.
Rudich, J., Halevy, A. H. and Kedar, N., 1970, Interaction of gibberellin and SADH on growth and sex expression of muskmelon. J. American Soc. Hort. Sci., 97: 369-372.
Ruebenbaur, T. and Schultis, L., 1960, Zastosovanie selektywnego gemetocydu FW-450 dla chemeicznego kastrowania kwaito buraka. Hodwla Roslin Aklimat Nasiennicto, USSR, 4: 199-204.
Saini, S. S. and Davis, G. N., 1969, Male sterility in Allium cepa and some species hybrids. Economic Botany, 23: 37-44.
Salgare, S. A., 1995, Gametocidal effect of acrolein on ornamental chillies. Flora and Fauna, Jhansi, 1: 95-98.
Salmon, N., 1963, Behandlung von Beta vulgaris Tel Aviv., Israel. Ref. Chem. Zentralbl., 134: 152-162.
Satyanarayana, K. V. V., Rao, N. V. P. R. G, Rao, G. M. and Murthy, B. K., 1996, Effect of gametocides on pollen sterility and plant morphology in partial sterile CMS lines of rice. New Botanist, 23: 1-4.
Sawheney, V. K., 1981, Abnormalities in pepper (Capsicum annuum L.) flowers induced by gibberellic acid. Canadian J. Bot., 59: 8-16.
Sayeres, E. R., 1959, The effect of FW-450 as a gametocide on Sorhgum vulgare. Undergrad Spec. Probl. Rep. Univ. Illinois Ubrana, p. 19.
Sccles, G. S. and Evans, L. E., 1979, Pollen development in male sterile and cytoplasmic male sterile rye. Canadian J. Bot., 57: 2782-2790.
Schulz, P. J., Cross, J. W. and Almeida, E., 1993, Chemical agents that inhibit pollen development: effects of the phenylcinnoline carboxylates SC-105 and SC-1271 on the ultrastructure of developing wheat anthers (Triticum aestivum L.). Sexual Pl. Reprod., 6: 108-121.
Schuster, W., 1961, Untersuchungen uber kunstlich induzierte pollen sterillitiat bie sonnenblumen (Helianthus annuus L.). Z. pflanzenzucht., 46: 389-404.
Seetharam, A. and Kumari, P. K., 1975, Induction of male sterility by gibberellic acid in sunflower. Indian J. Genet. and Pl. Breed., 35: 136-138.
Seetharam, A. and Kusumakumari, P., 1974, Giberellic acid induced male sterility in sunflower. Sci. Cul., 40(9): 398-399.
Seetharam, A. and Kusumakumari, P., 1976, Histological studies on cytoplasmic and GA3 induced male sterility levels of sunflower. Indian J. Genet., 36: 342-344.
Shivaprasad Shetty, 1995, Effect of GA3 and cycocel on maturity, seed yield and quality in china aster. M. Sc. (Agri.) Thesis, Univ. Agric. Sci., Bangalore.
Shivaramaiah, 1985, Induction of male sterility using gametocides in common millet (Pannicum miliaceum L.) and little millet (Panicum miliure L.). M. Sc. (Agri.) Thesis, Univ. Agri Sci., Dharwad.
Simanenko, V. K., 1982, Anther and microspore development offertile and cytoplasmical male sterility (CMS) line of sunflower. Tristol. Geneta, 16(5): 34-41.
Simonenko, V. K. and Karpavich, E. V., 1979, The cytological expression of different types of male sterility in sunflower.r Referatrun VI Zhurnal, 5: 65-69.
Singh, A. K., 1999, Male gametocidal effect of synthetic detergent in rice. Indian J. Genet Pl. Bred., 59(3): 371-373.
Singh, S. P. and Hadley, H. H., 1961, Pollen abortion in cytoplasmic male sterile sorghum. Crop Sci., 1: 430-432.
Sink, K. C. and Gunesch, M. L., 1966, Gametocidal effects of sodium alpha, beta-dichloroisobutyrate (DCIB) on petunias. In Proc. American Soc. Hort. Sci., 88: 657-661.
Sprova, M., 1975, New data on male sterility in sunflower induced by gibberellic acid. Resteniev dni Nanki, 12(1): 10-17.
Sreedhar, R. V., 2003, Assessment of genetic variability in niger (Guizotia abyssinica Cass.) germplasm. M. Sc. (Agri.) Thesis, Univ. Agric. Sci., Dharwad.
Starnes, W. J. and Hadley, H. H., 1962, Some effects of the gametocide alpha, beta-dichloroisobutyrate on soybean. Crop Sci., 2: 305-310.
Stoskopf, N. C. and Law J., 1972, Some observations on ethrel as a tool for developing hybrid cereals. Canadian J. Pl. Sci., 52: 680-683.
Sundararajan, N., Nagaraju, S., Venkataraman, S. and Jayanah, M. H., 1972, Design and analysis of field experiments. Univ. Agri. Sci., Hebbal, Bangalore.
Suryanarayan Reddy, B. G., Pushpa, G., Satyam, B. A. and Prakash, K. S., 1983, Preliminary studies on gibberellic acid inducted male sterility in finger millet. Proceedings of National Seminar, Jan. 12-13, 1983, Univ. Agri. Sci., Bangalore, Ed. By Seehtaram and Shanlare Gowda.
Swarnalatha, V., 2004, Induction of male sterility and histological studies on induced male sterility in niger (Guizotia abyssinica Cass). M. Sc. (Agri.) Thesis, Univ. Agric. Sci., Dharwad.
Tschabold, E. E., Heim, D. R., Beck, J. R., Wright, F. L., Rainey, D. P., Terando, N. H. and Schwer, J. F., 1988, LY 195259, new chemical hybridizing agent for wheat. Crop Science, 28: 583-588.
Vear, F., 1981, Determination of sunflower lines useable as a combining ability testers according to their aptitude to be male sterilized with giberellin. Sunflower News., 5: 5-8.
Veer Kumar, G. V., 2002, Studies on genetic variability, floral biology, autogamy and histology of GA3 induced male sterility in niger. M. Sc. (Agri.) Thesis, Univ. Agric. Sci., Bangalore.
Verma, M. M. and Kumar, J., 1978, Ethrel a male gametocide that can replace the male sterility genes in barley. Euphytica, 27: 865-968.
Verma, R. B., Singh, G. N., 1978, Stuides on chemical induction of male sterility in bhendi (Abelmoschus esculentus Moench). Indian J. Agri. Res., 12(1): 22-24.
Verober, A. I. and Blankarskaya, T. E., 1978, Cytoembryological changes of male sterility in sunflower. I. Genet I Selektoiya rast. Tez. Dokl. Lemngred, VSSR (1977) 103-104, Udersa Univ. Ukranian SSR from Refertivnvi Zhumal, 5: 51-65.
Vittalaya Kini A., 1981, histological and histochemical studies in cytoplasmic gibberellic acid induced male sterile genes of sunflower. M. Sc. (Agri.) Thesis, Univ. Agri. Sci., Bangalore.
Walkof, C., 1959, In Prog. Rep. on FW-450 Chem. Gametocide. Rohm and Haas, Philadelphia, p. 9.
Wang, X. and Que, R. F., 1981, Induction of pollen sterility by ethylene and gametocides in rice. Acta Phytophysiol. Sin., 7: 381-383.
Wang, X., QUr, R. F., 1981, Induction o pollen sterility by ethylene and gametocide in rice. ActaPhyto. Sini, 7(4): 381-383.
Wang, X. I., Meiyu, Y. U. and Yao, F. D., 1995, Effect of CRMS on male sterility of rice plants. Chinese Rice Res. News., 3: 2-4.
Warmke, H. E. and Overman, M. A., 1972a, Cytoplasmic male sterility in sorghum I callose behaviour in the fertile and sterile anthers. J. Here., 63: 103-108.
Warmke, H. E. and Overman, M. A., 1972b, Cytoplasmic male sterility in sorghum-II, Tapetal behaviour in fertile and sterile anthers. J Here., 63: 228-233.
Wit, F., 1960, Chemically induced male sterility, a new tool in plant breeding. Euphytica, 9: 1-9.
Yaduraju, N. T., 2000, Recent Adv. Herb. Use Res., IARI, New Delhi, pp. 42-48.
Yogendra Sharma, Sharma, 2005, Chemical hybridizing agents (CHA)—a tool for hybrid seed production—review. Agril. Rev., 26(2): 114-123.
Yu, C., Hu, S., He, P., Sun, G., Zhang, C. Yu, Y., 2006, Inducing male sterility in Brassica napus L. by a sulphonylurea herbicide, itribenuronmethyl. Pl. Bred., 125(1): 61-64.
Yu, GuiRong, XU, Liyuan, Du, Wenping, Wang, Y. P., 2005, studies on the selection of wheat CHA and the cultivation of hybrid wheat. South West China J. Agri. Sci., 18(1): 29-32.
Zdrilko, A. F., 1967, Producing plants with male sterility by chemical means. Referat Zhumal Abstacts, 10: 55-75.

What is claimed is:

1. A method of obtaining seeds comprising clonal embryos comprising
collecting one or more seeds produced by a maternal plant, wherein the maternal plant is unable to be pollinated,
wherein the one or more seeds comprise a parthenogenically-derived embryo that is a clone of the maternal plant.

2. The method of claim 1, wherein the maternal plant is defective in at least one RNA dependent DNA methylation pathway gene.

3. The method of claim 2, wherein the RNA dependent DNA methylation pathway gene is AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NRPD1b (NUCLEAR POLYMERASE D 1b), NRPD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9). NO OTHER GENES

4. The method of claim 3, wherein the AGO4 allele is ago4-6 or ago4-1.

5. The method of claim 3, wherein the AGO6 allele is ago6-2.

6. The method of claim 3, wherein the AGO9 allele is 9-2, 9-3 or 9-4.

7. The method of claim 3, wherein the AGO8 allele is ago 8-1.

8. The method of claim 3, wherein the RDR2 allele is rdr2-1.

9. The method of claim 3, wherein the RDR6 allele is rdr6-15 or rdr6-11.

10. The method of claim 3, wherein the SGS3 allele is sgs3-11.

11. The method of claim 3, wherein the DRM2 allele is drm2-2.

12. The method of claim 3, wherein the MET1 allele is met1-7.

13. The method of claim 1, wherein the maternal plant is unable to self-pollinate.

14. The method of claim 13, wherein the maternal plant's ability to self-pollinate is disrupted physically, chemically or genetically.

15. The method of claim 14, wherein chemical disruption comprises exposure to a gametocide that abolishes pollen formation.

16. The method of claim 15, wherein the gametocide includes at least one of maleic hydrazide (1,2-dihydropyridazine, 3-6-dione) (MH), 2,4-dichlorophenoxyacetic acid (2,4-D), a-naphthalene acetic acid (NAA), and tri-iodobenzoic acid (TIBA).

17. The method of claim 14, wherein physical disruption comprises emasculating the maternal plant.

18. A method of obtaining seeds comprising clonal embryos comprising
a) collecting one or more seeds produced by a maternal plant, wherein the maternal plant is defective in at least one RNA dependent DNA methylation pathway gene; and
b) sorting the seeds to separate the seeds comprising clonal embryos from the seeds comprising non-clonal embryos;
wherein the maternal plant is pollinated prior to collecting the seeds, and
wherein the one or more seeds produced by the maternal plant comprise an embryo that is a clone of the maternal plant.

19. The method of claim 18, wherein sorting the seeds is based on phenotype or genotype.

20. The method of claim 19, wherein the phenotype comprises size, shape, color, or a combination thereof of the seeds.

21. The method of claim 18, wherein the sorting is performed manually.

22. The method of claim 18, wherein the sorting is performed automatically.

23. The method of claim 22, wherein the automatic sorting comprises a machine comprising an optical detector.

24. The method of claim 18, wherein the sorting is done visually.

25. A method of screening for maternal plants that produce seeds comprising parthenogenically-derived clonal embryos comprising
a) obtaining a maternal plant unable to pass on paternally-derived chromosomes to embryos, wherein an activity of a gene of interest in a RNA dependent DNA methylation pathway is silenced in the plant;
b) harvesting the seeds; and
c) determining whether the seeds comprise clonal embyros, wherein the presence of seeds comprising clonal embryos indicates the maternal plant can produce parthenogenically-derived clonal embryos.

26. The method of claim 25, wherein the activity of the gene of interest is silenced t using RNA interference.

27. The method of claim 25, wherein the gene of interest is AGO4 (ARGONAUTE 4), AGOG (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

28. A method of increasing the yield of seeds comprising parthenogenically-derived clonal embryos comprising
a) obtaining a maternal plant unable to pass on paternally-derived chromosomes to embryos, wherein an activity of a gene of interest in a RNA dependent DNA methylation pathway is silenced in the plant;
b) pollinating the maternal plant;
c) collecting seeds produced by the maternal plant;
d) sorting the seeds comprising parthenogenically-derived clonal embryos from seeds comprising non-clonal embryos.

29. The method of claim 28, wherein sorting seeds comprising parthenogenically-derived clonal embryos from seeds comprising non-clonal embryos is based on phenotype or genotype.

30. The method of claim 29, wherein sorting based on phenotype comprises determining the size, shape, color, or a combination thereof, of the seeds.

31. The method of claim 28, wherein the sorting is performed manually.

32. The method of claim 28, wherein the sorting is performed automatically.

33. The method of claim 32, wherein the automatic sorting comprises a machine comprising an optical detector.

34. The method of claim 28, wherein the sorting is done visually.

35. The method of claim 28, wherein the RNA dependent DNA methylation pathway gene is AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

36. A maternal plant comprising a construct, wherein the construct comprises a nucleic acid sequence that renders the maternal plant defective for RNA-dependent DNA methylation.

37. The maternal plant of claim 36, wherein the maternal plant produces seeds comprising parthenogenically-derived clonal embryos.

38. The maternal plant of claim 36, wherein the nucleic acid sequence silences activity of a RNA-dependent DNA methylation pathway gene.

39. The maternal plant of claim 36, wherein the RNA dependent DNA methylation pathway gene is AGO4 (ARGONAUTE 4), AGO6 (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).

40. A seed comprising a parthenogenically-derived clonal embryo comprising a defective RNA-dependent DNA methylation pathway gene.

41. The seed of claim 40, wherein the seed, when grown, produces seeds comprising clonal embryos.

42. The seed of claim 40, wherein the RNA dependent DNA methylation pathway gene is AGO4 (ARGONAUTE 4), AGOG (ARGONAUTE 6), AGO8 (ARGONAUTE 8), AGO9 (ARGONAUTE 9), CMT3 (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NPRD1b (NUCLEAR POLYMERASE D 1b), NPRD2 (NUCLEAR POLYMERASE D 2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9).
